SEQUENCE LISTING (239 sequences)

SEQ ID NO: 1 - RNA, 21 nt, Artificial Sequence
  siRNA forward
gggagcugauggccuaggaaa

SEQ ID NO: 2 - RNA, 21 nt, Artificial Sequence
  (6)...(8) 2′-O-methyl-modified
uccuaggccaucagcucccac

SEQ ID NO: 3 - RNA, 21 nt, Artificial Sequence
  (6) and (8) 2′-O-methyl-modified
uccuaggccaucagcucccac

SEQ ID NO: 4 - DNA, 33 bp, Artificial Sequence
  reporter forward
tggtagttggagctggtggcgtaggcaagagtg

SEQ ID NO: 5 - DNA, 33 bp, Artificial Sequence
  reporter reverse
cactcttgcctacgccaccagctccaactacca

SEQ ID NO: 6 - DNA, 33 bp, Artificial Sequence
  reporter forward
tggtagttggagctgatggcgtaggcaagagtg

SEQ ID NO: 7 - DNA, 33 bp, Artificial Sequence
  reporter reverse
cactcttgcctacgccatcagctccaactacca

SEQ ID NO: 8 - DNA, 21 bp, Artificial Sequence
  siRNA forward
guuggagcugauggcguagtt

SEQ ID NO: 9 - DNA, 21 bp, Artificial Sequence
  siRNA reverse
cuacgccaucagcuccaactt

SEQ ID NO: 10 - RNA, 21 nt, Artificial Sequence
  siRNA forward
uuggagcugauggcguaggca

SEQ ID NO: 11 - RNA, 21 nt, Artificial Sequence
  siRNA reverse
ccuacgccaucagcuccaacu

SEQ ID NO: 12 - RNA, 21 nt, Artificial Sequence
  siRNA forward
uggagcugauggcguaggcaa

SEQ ID NO: 13 - RNA, 21 nt, Artificial Sequence
  siRNA reverse
gccuacgccaucagcuccaac

SEQ ID NO: 14 - RNA, 21 nt, Artificial Sequence
  siRNA forward
gggagcugauggcguaggaaa

SEQ ID NO: 15 - RNA, 21 nt, Artificial Sequence
  siRNA reverse
uccuacgccaucagcucccac

SEQ ID NO: 16 - RNA, 21 nt, Artificial Sequence
  siRNA forward
gggagcugauggcguaggaaa

SEQ ID NO: 17 - RNA, 21 nt, Artificial Sequence
  siRNA reverse (2)...(5) 2′-O-methyl modified
uccuacgccaucagcucccac

SEQ ID NO: 18 - RNA, 21 nt, Artificial Sequence
  siRNA forward
gggagcugauggcguaggaaa

SEQ ID NO: 19 - RNA, 21 nt, Artificial Sequence
  siRNA reverse (6)...(8) 2′-O-methyl modified
uccuacgccaucagcucccac

SEQ ID NO: 20 - RNA, 21 nt, Artificial Sequence
  siRNA forward
gggagcugauggcguacgaaa

SEQ ID NO: 21 - RNA, 21 nt, Artificial Sequence
  siRNA reverse
ucguacgccaucagcucccac

SEQ ID NO: 22 - RNA, 21 nt, Artificial Sequence
  siRNA forward
gggagcugauggcguuggaaa

SEQ ID NO: 23 - RNA, 21 nt, Artificial Sequence
  siRNA reverse
uccaacgccaucagcucccac

SEQ ID NO: 24 - RNA, 21 nt, Artificial Sequence
  siRNA forward
gggagcugauggcgaaggaaa

SEQ ID NO: 25 - RNA, 21 nt, Artificial Sequence
  siRNA reverse
uccuucgccaucagcucccac

SEQ ID NO: 26 - RNA, 21 nt, Artificial Sequence
  siRNA forward
gggagcugauggccuaggaaa

SEQ ID NO: 27 - RNA, 21 nt, Artificial Sequence
  siRNA reverse
uccuaggccaucagcucccac

SEQ ID NO: 28 - RNA, 21 nt, Artificial Sequence
  siRNA forward
gggagcugaugggguaggaaa

SEQ ID NO: 29 - RNA, 21 nt, Artificial Sequence
  siRNA reverse
uccuaccccaucagcucccac

SEQ ID NO: 30 - RNA, 21 nt, Artificial Sequence
  siRNA forward
gggagcugauggcguacgaaa

SEQ ID NO: 31 - RNA, 21 nt, Artificial Sequence
  siRNA reverse (6)...(8) 2′-O-methyl modified
ucguacgccaucagcucccac

SEQ ID NO: 32 - RNA, 21 nt, Artificial Sequence
  siRNA forward
gggagcugauggcguuggaaa

SEQ ID NO: 33 - RNA, 21 nt, Artificial Sequence
  siRNA reverse (6)...(8) 2′-O-methly modified
uccaacgccaucagcucccac

SEQ ID NO: 34 - RNA, 21 nt, Artificial Sequence
  siRNA
gggagcugauggcgaaggaaa

SEQ ID NO: 35 - RNA, 21 nt, Artificial Sequence
  siRNA reverse (6)...(8) 2′-O-methyl modified
uccuucgccaucagcucccac

SEQ ID NO: 36 - RNA, 21 nt, Artificial Sequence
  siRNA forward
gggagcugauggccuaggaaa

SEQ ID NO: 37 - RNA, 21 nt, Artificial Sequence
  siRNA reverse (6)...(8) 2′-O-methyl modified
uccuaggccaucagcucccac

SEQ ID NO: 38 - RNA, 21 nt, Artificial Sequence
  siRNA forward
gggagcugaugggguaggaaa

SEQ ID NO: 39 - RNA, 21 nt, Artificial Sequence
  siRNA reverse (6)...(8) 2′-O-methyl modified
uccuaccccaucagcucccac

SEQ ID NO: 40 - DNA, 33 bp, Artificial Sequence
  reporter forward
tggtagttggagctgttggcgtaggcaagagtg

SEQ ID NO: 41 - DNA, 33 bp, Artificial Sequence
  reporter reverse
cactcttgcctacgccaacagctccaactacca

SEQ ID NO: 42 - DNA, 33 bp, Artificial Sequence
  reporter forward
tggtagttggagctgctggcgtaggcaagagtg

SEQ ID NO: 43 - DNA, 33 bp, Artificial Sequence
  reporter reverse
cactcttgcctacgccagcagctccaactacca

SEQ ID NO: 44 - RNA, 21 nt, Artificial Sequence
  siRNA forward
gggagcuguuggcguaggaaa

SEQ ID NO: 45 - RNA, 21 nt, Artificial Sequence
  siRNA reverse
uccuacgccaacagcucccac

SEQ ID NO: 46 - RNA, 21 nt, Artificial Sequence
  siRNA forward
gggagcuguuggcgaaggaaa

SEQ ID NO: 47 - RNA, 21 nt, Artificial Sequence
  siRNA reverse (6)...(8) 2′-O-methyl modified
uccuucgccaacagcucccac

SEQ ID NO: 48 - RNA, 21 nt, Artificial Sequence
  siRNA forward
gggagcuguuggccuaggaaa

SEQ ID NO: 49 - RNA, 21 nt, Artificial Sequence
  siRNA reverse (6)...(8) 2′-O-methyl modified
uccuaggccaacagcucccac

SEQ ID NO: 50 - RNA, 21 nt, Artificial Sequence
  siRNA forward
gggagcugcuggcguaggaaa

SEQ ID NO: 51 - RNA, 21 nt, Artificial Sequence
  siRNA reverse
uccuacgccagcagcucccac

SEQ ID NO: 52 - RNA, 21 nt, Artificial Sequence
  siRNA forward
gggagcugcuggcgaaggaaa

SEQ ID NO: 53 - RNA, 21 nt, Artificial Sequence
  siRNA reverse (6)...(8) 2′-O-methyl modified
uccuucgccagcagcucccac

SEQ ID NO: 54 - RNA, 21 nt, Artificial Sequence
  siRNA forward
gggagcugcuggccuaggaaa

SEQ ID NO: 55 - RNA, 21 nt, Artificial Sequence
  siRNA reverse (6)...(8) 2′-O-methyl modified
uccuaggccagcagcucccac

SEQ ID NO: 56 - DNA, 40 bp, Artificial Sequence
  reporter forward
actggtggtggttggagcaggtggtgttgggaaaagcgca

SEQ ID NO: 57 - DNA, 40 bp, Artificial Sequence
  reporter reverse
tgcgcttttcccaacaccaccagctccaaccaccaccagt

SEQ ID NO: 58 - DNA, 40 bp, Artificial Sequence
  reporter forward
actggtggtggttggagcagatggtgttgggaaaagcgca

SEQ ID NO: 59 - DNA, 40 bp, Artificial Sequence
  reporter reverse
tgcgcttttcccaacaccatcagctccaaccaccaccagt

SEQ ID NO: 60 - RNA, 21 nt, Artificial Sequence
  siRNA forward
uggagcaggugguguugggaa

SEQ ID NO: 61 - RNA, 21 nt, Artificial Sequence
  siRNA reverse
cccaacaccaccugcuccaac

SEQ ID NO: 62 - RNA, 21 nt, Artificial Sequence
  siRNA forward
uggagcagaugguguugggaa

SEQ ID NO: 63 - RNA, 21 nt, Artificial Sequence
  siRNA reverse
cccaacaccaucugcuccaac

SEQ ID NO: 64 - RNA, 21 nt, Artificial Sequence
  siRNA forward
cggagcagauggugauggaaa

SEQ ID NO: 65 - RNA, 21 nt, Artificial Sequence
  siRNA reverse (6)...(8) 2′-O-methyl modified
uccaucaccaucugcuccgac

SEQ ID NO: 66 - DNA, 38 bp, Artificial Sequence
  reporter forward
catactggatacagctggacaagaagagtacagtgcca

SEQ ID NO: 67 - DNA, 38 bp, Artificial Sequence
  reporter reverse
tggcactgtactcttcttgtccagctgtatccagtatg

SEQ ID NO: 68 - DNA, 38 bp, Artificial Sequence
  reporter forward
catactggatacagctggacgagaagagtacagtgcca

SEQ ID NO: 69 - DNA, 38 bp, Artificial Sequence
  reporter reverse
tggcactgtactcttctcgtccagctgtatccagtatg

SEQ ID NO: 70 - RNA, 21 nt, Artificial Sequence
  siRNA forward
agcuggacaagaagaguacag

SEQ ID NO: 71 - RNA, 21 nt, Artificial Sequence
  siRNA reverse
guacucuucuuguccagcugu

SEQ ID NO: 72 - RNA, 21 nt, Artificial Sequence
  siRNA forward
agcuggacgagaagaguacag

SEQ ID NO: 73 - RNA, 21 nt, Artificial Sequence
  siRNA reverse
guacucuucucguccagcugu

SEQ ID NO: 74 - RNA, 21 nt, Artificial Sequence
  siRNA forward
ggcuggacgagaaguguaaag

SEQ ID NO: 75 - RNA, 21 nt, Artificial Sequence
  siRNA reverse (6)...(8) 2′-O-methyl modified
uuacacuucucguccagccgu

SEQ ID NO: 76 - DNA, 47 bp, Artificial Sequence
  reporter sense strand
tcgaggttaagagatccccacagtacttcaacgctagaagaacacag

SEQ ID NO: 77 - DNA, 47 bp, Artificial Sequence
  reporter antisense strand
aattctgtgttcttctagcgttgaagtactgtggggatctcttaacc

SEQ ID NO: 78 - DNA, 47 bp, Artificial Sequence
  reporter sense strand
tcgaggttaagagatccccacagtaattcaacgctagaagaacacag

SEQ ID NO: 79 - DNA, 47 bp, Artificial Sequence
  reporter antisense strand
aattctgtgttcttctagcgttgaattactgtggggatctcttaacc

SEQ ID NO: 80 - DNA, 47 bp, Artificial Sequence
  reporter sense strand
tcgagccggagcctttggaagtctgcgcccttgtgccctgcctccag

SEQ ID NO: 81 - DNA, 47 bp, Artificial Sequence
  reporter antisense strand
aattctggaggcagggcacaagggcgcagacttccaaaggctccggc

SEQ ID NO: 82 - DNA, 47 bp, Artificial Sequence
  reporter sense strand
tcgagccggagcctttggaagtctgtgcccttgtgccctgcctccag

SEQ ID NO: 83 - DNA, 47 bp, Artificial Sequence
  reporter antisense strand
aattctggaggcagggcacaagggcacagacttccaaaggctccggc

SEQ ID NO: 84 - DNA, 47 bp, Artificial Sequence
  reporter sense strand
tcgagcccacgcctgctccctcatctactgtgtgcacttcatcctgg

SEQ ID NO: 85 - DNA, 47 bp, Artificial Sequence
  reporter antisense strand
aattccaggatgaagtgcacacagtagatgagggagcaggcgtgggc

SEQ ID NO: 86 - DNA, 47 bp, Artificial Sequence
  reporter sense strand
tcgagcccacgcctgctccctcatccactgtgtgcacttcatcctgg

SEQ ID NO: 87 - DNA, 47 bp, Artificial Sequence
  reporter antisense strand
aattccaggatgaagtgcacacagtggatgagggagcaggcgtgggc

SEQ ID NO: 88 - DNA, 47 bp, Artificial Sequence
  reporter sense strand
tcgagagcagcagcagcagcagcagggggacctatcaggacagagtg

SEQ ID NO: 89 - DNA, 47 bp, Artificial Sequence
  reporter antisense strand
aattcactctgtcctgataggtccccctgctgctgctgctgctgctc

SEQ ID NO: 90 - DNA, 47 bp, Artificial Sequence
  reporter sense strand
tcgagagcagcagcagcagcagcagcgggacctatcaggacagagtg

SEQ ID NO: 91 - DNA, 47 bp, Artificial Sequence
  reporter antisense strand
aattcactctgtcctgataggtcccgctgctgctgctgctgctgctc

SEQ ID NO: 92 - DNA, 47 bp, Artificial Sequence
  reporter sense strand
tcgagtgctggagaccatccactggcgtgggcagatccgtcatttcg

SEQ ID NO: 93 - DNA, 47 bp, Artificial Sequence
  reporter antisense strand
aattcgaaatgacggatctgcccacgccagtggatggtctccagcac

SEQ ID NO: 94 - DNA, 47 bp, Artificial Sequence
  reporter sense strand
tcgagtgctggagaccatccactggtgtgggcagatccgtcatttcg

SEQ ID NO: 95 - DNA, 47 bp, Artificial Sequence
  reporter antisense strand
aattcgaaatgacggatctgcccacagcagtggatggtctccagcac

SEQ ID NO: 96 - DNA, 47 bp, Artificial Sequence
  reporter sense strand
tcgaggaatccattctggtgccactaccacagctccttctctgagtg

SEQ ID NO: 97 - DNA, 47 bp, Artificial Sequence
  reporter antisense strand
aattcactcagagaaggagctgtggtagtggcaccagaatggattcc

SEQ ID NO: 98 - DNA, 47 bp, Artificial Sequence
  reporter sense strand
tcgaggaatccattctggtgccactgccacagctccttctctgagtg

SEQ ID NO: 99 - DNA, 47 bp, Artificial Sequence
  reporter antisense strand
aattcactcagagaaggagctgtggcagtggcaccagaatggattcc

SEQ ID NO: 100 - DNA, 47 bp, Artificial Sequence
  reporter sense strand
tcgagaccccgccgcctgctgctgacgggcacaccgctgcagaacag

SEQ ID NO: 101 - DNA, 47 bp, Artificial Sequence
  reporter antisense strand
aattctgttctgcagcggtgtgcccgtcagcagcaggcggcggggtc

SEQ ID NO: 102 - DNA, 47 bp, Artificial Sequence
  reporter sense strand
tcgagaccccgccgcctgctgctgatgggcacaccgctgcagaacag

SEQ ID NO: 103 - DNA, 47 bp, Artificial Sequence
  reporter antisense strand
aattctgttctgcagcggtgtgcccatcagcagcaggcggcggggtc

SEQ ID NO: 104 - DNA, 47 bp, Artificial Sequence
  reporter sense strand
tcgagtggccccaatcggcctggtgctcatcgtgggaggctacttcg

SEQ ID NO: 105 - DNA, 47 bp, Artificial Sequence
  reporter antisense strand
aattcgaagtagcctcccacgatgagcaccaggccgattggggccac

SEQ ID NO: 106 - DNA, 47 bp, Artificial Sequence
  reporter sense strand
tcgagtggccccaatcggcctggtgttcatcgtgggaggctacttcg

SEQ ID NO: 107 - DNA, 47 bp, Artificial Sequence
  reporter antisense strand
aattcgaagtagcctcccacgatgaacaccaggccgattggggccac

SEQ ID NO: 108 - DNA, 47 bp, Artificial Sequence
  reporter sense strand
tcgagcaaggccttgcctggcttccgcaacttacacgtggacgaccg

SEQ ID NO: 109 - DNA, 47 bp, Artificial Sequence
  reporter antisense strand
aattcggtcgtccacgtgtaagttgcggaagccaggcaaggccttgc

SEQ ID NO: 110 - DNA, 47 bp, Artificial Sequence
  reporter sense strand
tcgagcaaggccttgcctggcttcctcaacttacacgtggacgaccg

SEQ ID NO: 111 - DNA, 47 bp, Artificial Sequence
  reporter antisense strand
aattcggtcgtccacgtgtaagttgaggaagccaggcaaggccttgc

SEQ ID NO: 112 - DNA, 47 bp, Artificial Sequence
  reporter sense strand
tcgaggaatcaatcgcatcttccacgagcggttcccatttgagctgg

SEQ ID NO: 113 - DNA, 47 bp, Artificial Sequence
  reporter antisense strand
aattccagctcaaatgggaaccgctcgtggaagatgcgattgattcc

SEQ ID NO: 114 - DNA, 47 bp, Artificial Sequence
  reporter sense strand
tcgaggaatcaatcgcatcttccacaagcggttcccatttgagctgg

SEQ ID NO: 115 - DNA, 47 bp, Artificial Sequence
  reporter antisense strand
aattccagctcaaatgggaaccgcttgtggaagatgcgattgattcc

SEQ ID NO: 116 - DNA, 47 bp, Artificial Sequence
  reporter sense strand
tcgagtcaagaccctcaacaacaagtttgcctccttcatcgacaagg

SEQ ID NO: 117 - DNA, 47 bp, Artificial Sequence
  reporter antisense strand
aattccttgtcgatgaaggaggcaaacttgttgttgagggtcttgac

SEQ ID NO: 118 - DNA, 47 bp, Artificial Sequence
  reporter sense strand
tcgagtcaagaccctcaacaacaaggttgcctccttcatcgacaagg

SEQ ID NO: 119 - DNA, 47 bp, Artificial Sequence
  reporter antisense strand
aattccttgtcgatgaaggaggcaaccttgttgttgagggtcttgac

SEQ ID NO: 120 - DNA, 47 bp, Artificial Sequence
  reporter sense strand
tcgagtctccgaagcccacacgtgtatccctgagaacaacggaggcg

SEQ ID NO: 121 - DNA, 47 bp, Artificial Sequence
  reporter antisense strand
aattcgcctccgttgttctcagggatacacgtgtgggcttcggagac

SEQ ID NO: 122 - DNA, 47 bp, Artificial Sequence
  reporter sense strand
tcgagtctccgaagcccacacgtgtgtccctgagaacaacggaggcg

SEQ ID NO: 123 - DNA, 47 bp, Artificial Sequence
  reporter antisense strand
aattcgcctccgttgttctcagggacacacgtgtgggcttcggagac

SEQ ID NO: 124 - DNA, 47 bp, Artificial Sequence
  reporter sense strand
tcgagcttaccgcagcttcagcaactccctgagccagtcaccgtgtg

SEQ ID NO: 125 - DNA, 47 bp, Artificial Sequence
  reporter antisense strand
aattcacacggtgactggctcagggagttgctgaagctgcggtaagc

SEQ ID NO: 126 - DNA, 47 bp, Artificial Sequence
  reporter sense strand
tcgagcttaccgcagcttcagcaaccccctgagccagtcaccgtgtg

SEQ ID NO: 127 - DNA, 47 bp, Artificial Sequence
  reporter antisense strand
aattcacacggtgactggctcagggggttgctgaagctgcggtaagc

SEQ ID NO: 128 - DNA, 47 bp, Artificial Sequence
  reporter sense strand
tcgagggataatatcaaacacgtcccgggaggcggcagtgtgcaaag

SEQ ID NO: 129 - DNA, 47 bp, Artificial Sequence
  reporter antisense strand
aattctttgcacactgccgcctcccgggacgtgtttgatattatccc

SEQ ID NO: 130 - DNA, 47 bp, Artificial Sequence
  reporter sense strand
tcgagggataatatcaaacacgtcctgggaggcggcagtgtgcaaag

SEQ ID NO: 131 - DNA, 47 bp, Artificial Sequence
  reporter antisense strand
aattctttgcacactgccgcctcccaggacgtgtttgatattatccc

SEQ ID NO: 132 - DNA, 47 bp, Artificial Sequence
  reporter sense strand
tcgagtgagttggaagacccaggggaaatgtctcctcccattgattg

SEQ ID NO: 133 - DNA, 47 bp, Artificial Sequence
  reporter antisense strand
aattcaatcaatgggaggagacatttcccctgggtcttccaactcac

SEQ ID NO: 134 - DNA, 47 bp, Artificial Sequence
  reporter sense strand
tcgagtgagttggaagacccagggggaatgtctcctcccattgattg

SEQ ID NO: 135 - DNA, 47 bp, Artificial Sequence
  reporter antisense strand
aattcaatcaatgggaggagacattccccctgggtcttccaactcac

SEQ ID NO: 136 - DNA, 47 bp, Artificial Sequence
  reporter sense strand
tcgagggcggcggcagcagcagcgggggctgcgggcggtcggggctg

SEQ ID NO: 137 - DNA, 47 bp, Artificial Sequence
  reporter antisense strand
aattcagccccgaccgcccgcagcccccgctgctgctgccgccgccc

SEQ ID NO: 138 - DNA, 47 bp, Artificial Sequence
  reporter sense strand
tcgagggcggcggcagcagcagcggcggctgcgggcggtcggggctg

SEQ ID NO: 139 - DNA, 47 bp, Artificial Sequence
  reporter antisense strand
aattcagccccgaccgcccgcagccgccgctgctgctgccgccgccc

SEQ ID NO: 140 - DNA, 47 bp, Artificial Sequence
  reporter sense strand
tcgaggacgggtgtggtacgcagccccttcgagtacccacagtactg

SEQ ID NO: 141 - DNA, 47 bp, Artificial Sequence
  reporter antisense strand
aattcagtactgtgggtactcgaaggggctgcgtaccacacccgtcc

SEQ ID NO: 142 - DNA, 47 bp, Artificial Sequence
  reporter sense strand
tcgaggacgggtgtggtacgcagccacttcgagtacccacagtactg

SEQ ID NO: 143 - DNA, 47 bp, Artificial Sequence
  reporter antisense strand
aattcagtactgtgggtactcgaagtggctgcgtaccacacccgtcc

SEQ ID NO: 144 - DNA, 47 bp, Artificial Sequence
  reporter sense strand
tcgagcggagacgagccaggtggcctcggcctaagacctgcctaggg

SEQ ID NO: 145 - DNA, 47 bp, Artificial Sequence
  reporter antisense strand
aattccctaggcaggtcttaggccgaggccacctggctcgtctccgc

SEQ ID NO: 146 - DNA, 47 bp, Artificial Sequence
  reporter sense strand
tcgagtgagaacattcagagttctccgagcattgaagacgatttcag

SEQ ID NO: 147 - DNA, 47 bp, Artificial Sequence
  reporter antisense strand
aattctgaaatcgtcttcaatgctcggagaactctgaatgttctcac

SEQ ID NO: 148 - DNA, 47 bp, Artificial Sequence
  reporter sense strand
tcgagtgagaacattcagagttctctgagcattgaagacgatttcag

SEQ ID NO: 149 - DNA, 47 bp, Artificial Sequence
  reporter antisense strand
aattctgaaatcgtcttcaatgctcagagaactctgaatgttctcac

SEQ ID NO: 150 - DNA, 47 bp, Artificial Sequence
  reporter sense strand
tcgagcttgaattccaagagcacacggtcttcagtgaagctgcaggg

SEQ ID NO: 151 - DNA, 47 bp, Artificial Sequence
  reporter antisense strand
aattccctgcagcttcactgaagaccgtgtgctcttggaattcaagc

SEQ ID NO: 152 - DNA, 47 bp, Artificial Sequence
  reporter sense strand
tcgagcttgaattccaagagcacacagtcttcagtgaagctgcaggg

SEQ ID NO: 153 - DNA, 47 bp, Artificial Sequence
  reporter antisense strand
aattccctgcagcttcactgaagactgtgtgctcttggaattcaagc

SEQ ID NO: 154 - DNA, 47 bp, Artificial Sequence
  reporter sense strand
tcgagaccgccgaagcctcagcccacgacccggaccccgcctcagtg

SEQ ID NO: 155 - DNA, 47 bp, Artificial Sequence
  reporter antisense strand
aattcactgaggcggggtccgggtcgtgggctgaggcttcggcggtc

SEQ ID NO: 156 - DNA, 47 bp, Artificial Sequence
  reporter sense strand
tcgagaccgccgaagcctcagcccaggacccggaccccgcctcagtg

SEQ ID NO: 157 - DNA, 47 bp, Artificial Sequence
  reporter antisense strand
aattcactgaggcggggtccgggtcctgggctgaggcttcggcggtc

SEQ ID NO: 158 - DNA, 47 bp, Artificial Sequence
  reporter sense strand
tcgagtgttccgggccctgggcctgcggcacctggtggtggtggacg

SEQ ID NO: 159 - DNA, 47 bp, Artificial Sequence
  reporter antisense strand
aattcgtccaccaccaccaggtgccgcaggcccagggcccggaacac

SEQ ID NO: 160 - DNA, 47 bp, Artificial Sequence
  reporter sense strand
tcgagtgttccgggccctgggcctgtggcacctggtggtggtggacg

SEQ ID NO: 161 - DNA, 47 bp, Artificial Sequence
  reporter antisense strand
aattcgtccaccaccaccaggtgccacaggcccagggcccggaacac

SEQ ID NO: 162 - DNA, 47 bp, Artificial Sequence
  reporter sense strand
tcgagtcccctccatcatgaatgtgctgctggtgtgtctcatcttcg

SEQ ID NO: 163 - DNA, 47 bp, Artificial Sequence
  reporter antisense strand
aattcgaagatgagacacaccagcagcacattcatgatggaggggac

SEQ ID NO: 164 - DNA, 47 bp, Artificial Sequence
  reporter sense strand
tcgagtcccctccatcatgaatgtggtgctggtgtgtctcatcttcg

SEQ ID NO: 165 - DNA, 47 bp, Artificial Sequence
  reporter antisense strand
aattcgaagatgagacacaccagcaccacattcatgatggaggggac

SEQ ID NO: 166 - DNA, 47 bp, Artificial Sequence
  reporter sense strand
tcgagacatcattggtgcctccagcgactgcagcacctgctttgtgg

SEQ ID NO: 167 - DNA, 47 bp, Artificial Sequence
  reporter antisense strand
aattccacaaagcaggtgctgcagtcgctggaggcaccaatgatgtc

SEQ ID NO: 168 - DNA, 47 bp, Artificial Sequence
  reporter sense strand
tcgagacatcattggtgcctccagctactgcagcacctgctttgtgg

SEQ ID NO: 169 - DNA, 47 bp, Artificial Sequence
  reporter antisense strand
aattccacaaagcaggtgctgcagtagctggaggcaccaatgatgtc

SEQ ID NO: 170 - DNA, 47 bp, Artificial Sequence; identical to SEQ ID NO: 116
  reporter sense strand

SEQ ID NO: 171 - DNA, 47 bp, Artificial Sequence; identical to SEQ ID NO: 117
  reporter antisense strand

SEQ ID NO: 172 - DNA, 47 bp, Artificial Sequence; identical to SEQ ID NO: 118
  reporter sense strand

SEQ ID NO: 173 - DNA, 47 bp, Artificial Sequence; identical to SEQ ID NO: 119
  reporter antisense strand

SEQ ID NO: 174 - RNA, 21 nt, Artificial Sequence
  siRNA
ccacaguacuucaacgcuaga

SEQ ID NO: 175 - RNA, 21 nt, Artificial Sequence
  siRNA
uagcguugaaguacugugggg

SEQ ID NO: 176 - RNA, 21 nt, Artificial Sequence
  siRNA
gaagucugcgcccuugugccc

SEQ ID NO: 177 - RNA, 21 nt, Artificial Sequence
  siRNA
gcacaagggcgcagacuucca

SEQ ID NO: 178 - RNA, 21 nt, Artificial Sequence
  siRNA
cccucaucuacugugugcacu

SEQ ID NO: 179 - RNA, 21 nt, Artificial Sequence
  siRNA
ugcacacaguagaugagggag

SEQ ID NO: 180 - RNA, 21 nt, Artificial Sequence
  siRNA
agcagcagggggaccuaucag

SEQ ID NO: 181 - RNA, 21 nt, Artificial Sequence
  siRNA
agcagcagggggaccuaucag

SEQ ID NO: 182 - RNA, 21 nt, Artificial Sequence
  siRNA
uccacuggcgugggcagaucc

SEQ ID NO: 183 - RNA, 21 nt, Artificial Sequence
  siRNA
aucugcccacgccaguggaug

SEQ ID NO: 184 - RNA, 21 nt, Artificial Sequence
  siRNA
gugccacuaccacagcuccuu

SEQ ID NO: 185 - RNA, 21 nt, Artificial Sequence
  siRNA
ggagcugugguaguggcacca

SEQ ID NO: 186 - RNA, 21 nt, Artificial Sequence
  siRNA
gcugcugacgggcacaccgcu

SEQ ID NO: 187 - RNA, 21 nt, Artificial Sequence
  siRNA
cggugugcccgucagcagcag

SEQ ID NO: 188 - RNA, 21 nt, Artificial Sequence
  siRNA
gccuggugcucaucgugggag

SEQ ID NO: 189 - RNA, 21 nt, Artificial Sequence
  siRNA
cccacgaugagcaccaggccg

SEQ ID NO: 190 - RNA, 21 nt, Artificial Sequence
  siRNA
gcacaguaauucaucgcuaga

SEQ ID NO: 191 - RNA, 21 nt, Artificial Sequence
  siRNA
uagcgaugaauuacugugcgg

SEQ ID NO: 192 - RNA, 21 nt, Artificial Sequence
  siRNA
gaagucugugcccaugugacc

SEQ ID NO: 193 - RNA, 21 nt, Artificial Sequence
  siRNA
ucacaugggcacagacuucca

SEQ ID NO: 194 - RNA, 21 nt, Artificial Sequence
  siRNA
gccucauccacugagugcacu

SEQ ID NO: 195 - RNA, 21 nt, Artificial Sequence
  siRNA
ugcacucaguggaugaggcag

SEQ ID NO: 196 - RNA, 21 nt, Artificial Sequence
  siRNA
ggcagcagcgggagcuauaag

SEQ ID NO: 197 - RNA, 21 nt, Artificial Sequence
  siRNA
uauagcucccgcugcugccgc

SEQ ID NO: 198 - RNA, 21 nt, Artificial Sequence
  siRNA
gccacugguguggccagaacc

SEQ ID NO: 199 - RNA, 21 nt, Artificial Sequence
  siRNA
uucuggccacaccaguggcug

SEQ ID NO: 200 - RNA, 21 nt, Artificial Sequence
  siRNA
gugccacugccacugcucauu

SEQ ID NO: 201 - RNA, 21 nt, Artificial Sequence
  siRNA
ugagcaguggcaguggcacca

SEQ ID NO: 202 - RNA, 21 nt, Artificial Sequence
  siRNA
gcugcugaugggcucaccacu

SEQ ID NO: 203 - RNA, 21 nt, Artificial Sequence
  siRNA
uggugagcccaucagcagcag

SEQ ID NO: 204 - RNA, 21 nt, Artificial Sequence
  siRNA
gccugguguucaugguggaag

SEQ ID NO: 205 - RNA, 21 nt, Artificial Sequence
  siRNA
gccaccaugaacaccaggccg

SEQ ID NO: 206 - RNA, 21 nt, Artificial Sequence
  siRNA
gggcuuccucaacauacaagu

SEQ ID NO: 207 - RNA, 21 nt, Artificial Sequence
  siRNA
uuguauguugaggaagcccgg

SEQ ID NO: 208 - RNA, 21 nt, Artificial Sequence
  siRNA
gcuuccacaagcgcuuccaau

SEQ ID NO: 209 - RNA, 21 nt, Artificial Sequence
  siRNA
uggaagcgcuuguggaagcug

SEQ ID NO: 210 - RNA, 21 nt, Artificial Sequence
  siRNA
ggagcagccggccgagcuacg

SEQ ID NO: 211 - RNA, 21 nt, Artificial Sequence
  siRNA
uagcucggccggcugcuccuc

SEQ ID NO: 212 - RNA, 21 nt, Artificial Sequence
  siRNA
gcacgugugucccagagaaca

SEQ ID NO: 213 - RNA, 21 nt, Artificial Sequence
  siRNA
uucucugggacacacgugcgg

SEQ ID NO: 214 - RNA, 21 nt, Artificial Sequence
  siRNA
gcagcaacccccucagccagu

SEQ ID NO: 215 - RNA, 21 nt, Artificial Sequence
  siRNA
uggcugaggggguugcugcag

SEQ ID NO: 216 - RNA, 21 nt, Artificial Sequence
  siRNA
gcacguccugggacgcggaag

SEQ ID NO: 217 - RNA, 21 nt, Artificial Sequence
  siRNA
uccgcgucccaggacgugcuu

SEQ ID NO: 218 - RNA, 21 nt, Artificial Sequence
  siRNA
gccagggggaaugacuccacc

SEQ ID NO: 219 - RNA, 21 nt, Artificial Sequence
  siRNA
uggagucauucccccuggcuc

SEQ ID NO: 220 - RNA, 21 nt, Artificial Sequence
  siRNA
ggcagcggcggcuccgggagg

SEQ ID NO: 221 - RNA, 21 nt, Artificial Sequence
  siRNA
ucccggagccgccgcugccgc

SEQ ID NO: 222 - RNA, 21 nt, Artificial Sequence
  siRNA
gcgcagccacuuccaguaacc

SEQ ID NO: 223 - RNA, 21 nt, Artificial Sequence
  siRNA
uuacuggaaguggcugcgcac

SEQ ID NO: 224 - RNA, 21 nt, Artificial Sequence
  siRNA
ggguggccucggcguaagacc

SEQ ID NO: 225 - RNA, 21 nt, Artificial Sequence
  siRNA
ucuuacgccgaggccacccgg

SEQ ID NO: 226 - RNA, 21 nt, Artificial Sequence
  siRNA
gaguucucugagcuuugaaga

SEQ ID NO: 227 - RNA, 21 nt, Artificial Sequence
  siRNA
uucaaagcucagagaacucug

SEQ ID NO: 228 - RNA, 21 nt, Artificial Sequence
  siRNA
gagcacacagucuacaguaaa

SEQ ID NO: 229 - RNA, 21 nt, Artificial Sequence
  siRNA
uacuguagacugugugcucuu

SEQ ID NO: 230 - RNA, 21 nt, Artificial Sequence
  siRNA
gcagcccaggaccgggacacc

SEQ ID NO: 231 - RNA, 21 nt, Artificial Sequence
  siRNA
ugucccgguccugggcugcgg

SEQ ID NO: 232 - RNA, 21 nt, Artificial Sequence
  siRNA
ggacccggacgccguggacca

SEQ ID NO: 233 - RNA, 21 nt, Artificial Sequence
  siRNA
uccagcugccacaggccccgg

SEQ ID NO: 234 - RNA, 21 nt, Artificial Sequence
  siRNA
ggaauguggugcucguguauc

SEQ ID NO: 235 - RNA, 21 nt, Artificial Sequence
  siRNA
uacacgagcaccacauuccug

SEQ ID NO: 236 - RNA, 21 nt, Artificial Sequence
  siRNA
gcuccagcuacuggagcaacu

SEQ ID NO: 237 - RNA, 21 nt, Artificial Sequence
  siRNA
uugcuccaguagcuggagcca

SEQ ID NO: 238 - RNA, 21 nt, Artificial Sequence
  siRNA
gcaacaagguugcguccuaca

SEQ ID NO: 239 - RNA, 21 nt, Artificial Sequence
  siRNA
uaggacgcaaccuuguugcug

(19) United States
(12) Patent Application Publication
(10) Pub. No.: US 2024/0141337 A1
(43) Pub. Date: May 2, 2024

(54) RNA MOLECULE, CHIMERIC NA MOLECULE, DOUBLE-STRANDED RNA MOLECULE, AND DOUBLE-STRANDED CHIMERIC NA MOLECULE

(71) Applicant: THE UNIVERSITY OF TOKYO, Bunkyo-ku, Tokyo (JP)

(72) Inventors: Kumiko UI-TEI, Bunkyo-ku, Tokyo (JP); Yoshiaki KOBAYASHI, Bunkyo-ku, Tokyo (JP); Atsushi SATO, Bunkyo-ku, Tokyo (JP); Yoshimasa ASANO, Bunkyo-ku, Tokyo (JP); Yuria SUZUKI, Bunkyo-ku, Tokyo (JP); Naomi LEDEY, Bunkyo-ku, Tokyo (JP); Kaoru SAIGO, Tokyo (JP); Yukikazu NATORI, Kanagawa (JP)

(73) Assignee: THE UNIVERSITY OF TOKYO, Bunkyo-ku, Tokyo (JP)

(21) Appl. No.: 18/008,945

(22) PCT Filed: Jan. 17, 2022
(86) PCT No.: PCT/JP2022/001431

§ 371 Date: Dec. 7, 2022

(30) Foreign Application Priority Data
Jan. 15, 2021 (JP) 2021-005335

(51) Int. Cl. C12N 15/113

(52) U.S. Cl. CPC C12N 15/113; C12N 2310/14; C12N 2310/3231; C12N 2310/344

(57) ABSTRACT

RNA molecules for RNA interference to target a mutant allele with a point mutation, wherein the molecule has a nucleotide sequence complementary to a nucleotide sequence of a coding region of the mutant allele; and when counted from the base at the 5′-end in the nucleotide sequence complementary to the sequence of the mutant allele: a base at position 5 or 6 is mismatched with a base in the mutant allele; a base at position 10 or 11 is at the position of the point mutation and is identical to the base at the position of the point mutation in the mutant allele; the group at the 2′-position of the pentose in the ribonucleotide at position 8 is modified with OCH₃, halogen, or LNA; and the group at the 2′-position of the pentose in the ribonucleotide at position 7 is not modified with any of OCH₃, halogen, and LNA.

### TECHNICAL FIELD

The present invention relates to RNA molecules, chimeric NA molecules, double-stranded RNA molecules, and double-stranded chimeric NA molecules, for use in RNA interference.

### RELATED ART

RNA interference is a simple and efficient way to specifically suppress the expression of a given target gene in cells.

siRNA, however, has been understood to suppress the expression of unintended targets (off-targeted genes) more than expected at the beginning (Jackson, A. L. et al., (2003) Nature Biotechnology vol. 21, pp. 635-637).

Particularly, it has been shown that the stronger the affinity of siRNA to the mRNA of the target, the greater the off-target effects are induced (Ui-Tei, K. et al. (2008) Nucleic Acids Res. vol. 36, pp. 7100-7109).

### SUMMARY OF THE INVENTION

### Problem to be Solved by the Invention

An object of the present invention is to provide novel RNA molecules, novel chimeric NA molecules, novel double-stranded RNA molecules, and novel double-stranded chimeric NA molecules.

### Means to Solve the Problem

During intensive efforts directed toward identifying RNA sequences with reduced off-target effects, the present inventors found that, when the molecules have a mismatch at position 5 or 6 and are modified at the 2′-position of the pentose in each of the ribonucleotides at positions 6-8 or positions 7 and 8, the contribution of nucleotides at positions 10 and 11 in RNA molecules to off-target effects becomes lower. Thus, in RNA interference targeted to a mutant allele of a gene which has a point mutation relative to its corresponding wild-type allele, the present inventors succeeded in mainly suppressing the expression of the mutant allele but not substantially suppressing the expression of the wild-type allele, by designing an RNA molecule in which the base at position 10 or 11 is at the position of the point mutation and is identical to the one in the mutant allele, and using a double-stranded RNA molecule with the aforementioned RNA molecule as a guide strand for RNA interference. The present invention was thus completed.

An aspect of the present invention is an RNA molecule for use in RNA interference to target a mutant allele of a gene, the mutant allele having a point mutation relative to a wild-type allele of the gene, wherein the gene is selected from the group consisting of HTT, ATXN3, ZMYM3, CTNNB1, SMARCA4, SMO, AR, DNM2, KRT14, IL4R, MAPT, MS4A2, PABPNI, RHO, SCNIA, APOB, F12, CLCN7, SCN8A, PCSK9, and KRT6A, and wherein the RNA molecule satisfies the followings:
(1) the molecule has a nucleotide sequence complementary to a nucleotide sequence of a coding region of the mutant allele except for a base specified in (2-1) below; and
(2) when counted from the base at the 5′-end of a nucleotide sequence complementary to the nucleotide sequence of the mutant allele,
   (2-1) a base at position 5 or 6 is mismatched with a base in the mutant allele;
   (2-2) a base at position 10 or 11 is at the position of the point mutation and is identical to the base at the position of the point mutation in the mutant allele; and
   (2-3) the group at the 2′-position of the pentose in each of ribonucleotides at positions 6-8 or positions 7 and 8 is independently modified with OCH₃, halogen, or LNA. The halogen may be fluorine. When the base at the 5′-end of the nucleotide sequence specified in (1) above is not adenine or uracil, the base may be replaced by adenine or uracil. When the base at the 3′-end of the nucleotide sequence specified in (1) above is not cytosine or guanine, the base may be replaced by cytosine or guanine. Any of the aforementioned RNA molecules may contain 13-28 nucleotides. Any of the aforementioned RNA molecules may be a chimeric NA molecule wherein one or more ribonucleotides are each replaced by a deoxyribonucleotide, an artificial nucleic acid, or a nucleic acid analog.

A further aspect of the present invention is a double-stranded RNA molecule including a guide strand and a passenger strand, wherein the guide strand is any one of the aforementioned RNA molecules, and the passenger strand is an RNA molecule with a sequence complementary to that of the RNA molecule of the guide strand. An overhang may be present at the 3′-end of the guide strand and/or at the 3′-end of the passenger strand. Either or both of the overhangs may be 1-3 nucleotide(s) long. Any of the aforementioned double-stranded RNA molecules may be a double-stranded chimeric NA molecule in which one or more ribonucleotides are each replaced by a deoxyribonucleotide, an artificial nucleic acid, or a nucleic acid analog.

A further aspect of the present invention is a method for producing an RNA molecule for use as a guide strand in RNA interference, including the step of producing any one of the aforementioned RNA molecules. The RNA molecule may be a chimeric NA molecule wherein one or more ribonucleotides are each replaced by a deoxyribonucleotide, an artificial nucleic acid, or a nucleic acid analog.

A further aspect of the present invention is a method for performing RNA interference to target a mutant allele of a gene in a cell containing a wild-type allele of the gene and the mutant allele of the gene, the mutant allele having a point mutation, wherein the method includes the step of introducing any one of the aforementioned RNA molecules, chimeric NA molecules, double-stranded RNA molecules, or any one of the aforementioned double-stranded chimeric NA molecules into the cell.

A further aspect of the present invention is a therapeutic agent for a patient with a disease or a prophylactic agent for a carrier of the disease, the patient or the carrier having wild-type and mutant alleles of a causative gene for the disease, the mutant allele having a point mutation and being responsible for the disease, wherein the therapeutic agent includes, as an active ingredient, any one of the aforementioned RNA molecules, chimeric NA molecules, double-stranded RNA molecules, or any one of the aforementioned double-stranded chimeric NA molecules. The disease may be one of those listed in Table 1 below.

TABLE 1

| Type | Gene | Gene name | Diseases |
|---|---|---|---|
| Triplet | HTT | Huntingtin | Huntington's disease |
| repeat | ATXN3 | ataxin 3 | Machado-Joseph disease |
| diseases | | | (spinocerebellar ataxia-3) |
| Tumors | ZMYM3 | zinc finger MYM- | medulloblastoma, lung |
| | | type containing 3 | adenocarcinoma, lung |
| | | | squamous cell carcinoma, small |
| | | | cell lung cancer, and medullary |
| | | | thyroid carcinoma |
| | CTNNB1 | catenin beta 1 | non-small cell lung cancer |
| | | | (NSCLC), and hepatocellular |
| | | | carcinoma |
| | SMARCA4 | SWI/SNF related, | ovarian clear cell carcinoma, |
| | | matrix associated, | SMARCA4-deficient thoracic |
| | | actin dependent | sarcomatoid tumor, and lung |
| | | regulator of | tumor |
| | | chromatin, | |
| | | subfamily a, | |
| | | member 4 | |
| | SMO | smoothened, | basal cell carcinoma, and |
| | | frizzled class | medulloblastoma |
| | | receptor | |
| | AR | androgen receptor | prostate cancer, and salivary |
| | | | duct carcinoma |
| Genetic | DNM2 | dynamin 2 | centronuclear myopathy-1 |
| diseases | KRT14 | keratin 14 | congenital epidermolysis |
| | | | bullosa |
| | IL4R | interleukin 4 | atopic susceptibility to |
| | | receptor | disorders such as atopic |
| | | | dermatitis |
| | MAPT | microtubule | frontotemporal dementia |
| | | associated protein | (±parkinsonism symptoms), and |
| | | tau | Alzheimer's disease |
| | MS4A2 | membrane | atopic susceptibility to |
| | | spanning 4- | disorders such as atopic |
| | | domains A2 | dermatitis, and childhood |
| | | | asthma |
| | PABPN1 | poly(A) binding | oculopharyngeal muscular |
| | | protein nuclear 1 | dystrophy (OMPD) |
| | RHO | Rhodopsin | retinitis pigmentosa 4 |
| | SCNIA | sodium voltage- | early infantile epileptic |
| | | gated channel | encephalopathy 6 (Dravet |
| | | alpha subunit 1 | syndrome) |
| | APOB | apolipoprotein B | familial hypercholesterolemia, |
| | | | type 2 |
| | F12 | coagulation factor | hereditary angioedema, type III |
| | | XII | |
| | CLCN7 | chloride voltage- | autosomal dominant |
| | | gated channel 7 | osteopetrosis, type 2 |
| | SCN8A | sodium voltage- | developmental and epileptic |
| | | gated channel | encephalopathy, 13, and Dravet |
| | | alpha subunit 8 | syndrome |
| | PCSK9 | proprotein | familial hypercholesterolemia, |
| | | convertase | type 3 |
| | | subtilisin/kexin | |
| | | type 9 | |
| | KRT6A | keratin 6A | pachyonychia congenita, and |
| | | | epidermolysis bullosa |

A further aspect of the present invention is a method for selecting an RNA molecule, a chimeric NA molecule, a double-stranded RNA molecule, or a double-stranded chimeric NA molecule for use in RNA interference to silence a target gene, the method including the steps of evaluating a gene-specific silencing ability of a plurality of the RNA molecules, the chimeric NA molecules, the double-stranded RNA molecules, or the double-stranded chimeric NA molecules, to the target gene by performing the aforementioned RNAi-based method in vitro using each of the plurality of any one of the aforementioned RNA molecules, chimeric NA molecules, double-stranded RNA molecules, or any one of the aforementioned double-stranded chimeric NA molecules; and selecting an RNA molecule, a chimeric NA molecule, a double-stranded RNA molecule, or a double-stranded chimeric NA molecule having at least a certain level of the gene-specific silencing ability.

### BRIEF DESCRIPTION OF THE DRAWING

FIG. 1 shows graphs of the results on silencing abilities of siRNAs that target the K-ras gene, in each of which either one of positions 9-11 corresponded to the position of the point mutation, in an example of the present invention.

FIG. 2 shows graphs of the results on silencing abilities of an siRNA that targets the K-ras gene, in which position 11 corresponded to the position of the point mutation, the base at the 5′-end of the guide strand was changed from guanine to uracil, and the base at the 5′-end of the passenger strand was changed from uracil to guanine, in an example of the present invention.

FIG. 3 shows graphs of the results on silencing abilities of siRNAs that target the K-ras gene, in each of which position 11 corresponded to the position of the point mutation, the base at the 5′-end of the guide strand was changed from guanine to uracil, the base at the 5′-end of the passenger strand was changed from uracil to guanine, and the group at the 2′-position of the pentose in each of ribonucleotides at positions 6-8 of the guide strand was replaced by OCH₃, in an example of the present invention.

FIG. 4 shows graphs of the results on silencing abilities of siRNAs that target the K-ras gene, in each of which position 11 corresponded to the position of the point mutation, the base at the 5′-end of the guide strand was changed from guanine to uracil, the base at the 5′-end of the passenger strand was changed from uracil to guanine, and the base at either one of positions 3-7 of the guide strand was mismatched with that of the A-mutant allele, in an example of the present invention.

FIG. 5 shows graphs of the results on silencing abilities of siRNAs that target the K-ras gene, in each of which position 11 corresponded to the position of the point mutation, the base at the 5′-end of the guide strand was changed from guanine to uracil, the base at the 5′-end of the passenger strand was changed from uracil to guanine, the group at the 2′-position of the pentose in each of ribonucleotides at positions 6-8 of the guide strand was replaced by OCH₃, and the base at either one of positions 3-7 of the guide strand was mismatched with that of the A-mutant allele, in an example of the present invention.

FIG. 6 shows graphs of the results on silencing abilities of siRNAs specific for the A-mutant allele of the K-ras gene, to the wild-type allele, the T-mutant allele of the K-ras gene (c. 35G>T), and the C-mutant allele of the K-ras gene (c. 35 G>C), in an example of the present invention.

FIG. 7 shows graphs of the results on silencing abilities of siRNAs specific for the T-mutant allele of the K-ras gene, to the wild-type allele, and the A- and C-mutant alleles of the K-ras gene, in an example of the present invention.

FIG. 8 shows graphs of the results on silencing abilities of siRNAs specific for the C-mutant allele of the K-ras gene, to the wild-type allele, and the A- and T-mutant alleles of the K-ras gene, in an example of the present invention.

FIG. 9 shows graphs of the results on silencing abilities of siRNAs that target the point mutation in nt 35 of cDNA of the N-ras gene, in which position 11 corresponded to the position of the point mutation in the A-mutant allele of the N-ras gene (c. 35G>A) (hereinafter, referred to as the “A-mutant N35 allele”), the base at the 5′-end of the guide strand was changed from cytosine to uracil, the base at the 5′-end of the passenger strand was changed from adenine to guanine, the group at the 2′-position of the pentose in each of ribonucleotides at positions 6-8 of the guide strand was replaced by OCH₃, and the base at position 5 of the guide strand was mismatched with that of the A-mutant N35 allele, in an example of the present invention.

FIG. 10 shows graphs of the results on silencing abilities of siRNAs that target the point mutation in nt 182 of cDNA of the N-ras gene, in which position 11 corresponded to the position of the point mutation in the G-mutant allele of the N-ras gene (c. 182A>G) (hereinafter, referred to as the “G-mutant N182 allele”), the base at the 5′-end of the guide strand was changed from guanine to uracil, the base at the 5′-end of the passenger strand was changed from adenine to guanine, the group at the 2′-position of the pentose in each of ribonucleotides at positions 6-8 of the guide strand was replaced by OCH₃, and the base at position 5 of the guide strand was mismatched with that of the G-mutant N182 allele, in an example of the present invention.

FIG. 11 shows graphs of the results on silencing abilities of a first siRNA that targets the HTT gene, in which position 11 corresponded to the position of the point mutation, the base at the 5′-end of the passenger strand was changed from cytosine to guanine, the group at the 2′-position of the pentose in each of ribonucleotides at positions 6-8 of the guide strand was replaced by OCH₃, and the base at position 6 of the guide strand was mismatched by changing it from uracil to adenine, in an example of the present invention.

FIG. 12 shows graphs of the results on silencing abilities of a second siRNA that targets the HTT gene, in which position 11 corresponded to the position of the point mutation, the base at the 5′-end of the guide strand was changed from guanine to uracil, the group at the 2′-position of the pentose in each of ribonucleotides at positions 6-8 of the guide strand was replaced by OCH₃, and the base at position 6 of the guide strand was mismatched by changing it from adenine to uracil, in an example of the present invention.

FIG. 13 shows graphs of the results on silencing abilities of a third siRNA that targets the HTT gene, in which position 11 corresponded to the position of the point mutation, the base at the 5′-end of the passenger strand was changed from cytosine to guanine, the group at the 2′-position of the pentose in each of ribonucleotides at positions 6-8 of the guide strand was replaced by OCH₃, and the base at position 6 of the guide strand was mismatched by changing it from adenine to uracil, in an example of the present invention.

FIG. 14 shows graphs of the results on silencing abilities of siRNAs that target the ATXN 3 gene, in each of which position 11 corresponded to the position of the point mutation, the base at the 5′-end of the guide strand was changed from guanine to uracil, the base at the 5′-end of the passenger strand was changed from adenine to guanine, the group at the 2′-position of the pentose in each of ribonucleotides at positions 6-8 of the guide strand was replaced by OCH₃, and the base at position 6 of the guide strand was mismatched by changing it from guanine to cytosine, in an example of the present invention.

FIG. 15 shows graphs of the results on silencing abilities of siRNAs that target the ZMYM3 gene, in each of which position 11 corresponded to the position of the point mutation, the base at the 5′-end of the guide strand was changed from adenine to uracil, the base at the 5′-end of the passenger strand was changed from uracil to guanine, the group at the 2′-position of the pentose in each of ribonucleotides at positions 6-8 of the guide strand was replaced by OCH₃, and the base at position 6 of the guide strand was mismatched by changing it from cytosine to guanine, in an example of the present invention.

FIG. 16 shows graphs of the results on silencing abilities of siRNAs that target the CTNNB1 gene, in each of which position 11 corresponded to the position of the point mutation, the base at the 5′-end of the guide strand was changed from guanine to uracil, the group at the 2′-position of the pentose in each of ribonucleotides at positions 6-8 of the guide strand was replaced by OCH₃, and the base at position 6 of the guide strand was mismatched by changing it from uracil to adenine, in an example of the present invention.

FIG. 17 shows graphs of the results on silencing abilities of siRNAs that target the SMARCA4 gene, in each of which position 11 corresponded to the position of the point mutation, the base at the 5′-end of the guide strand was changed from cytosine to uracil, the group at the 2′-position of the pentose in each of ribonucleotides at positions 6-8 of the guide strand was replaced by OCH₃, and the base at position 6 of the guide strand was mismatched by changing it from uracil to adenine, in an example of the present invention.

FIG. 18 shows graphs of the results on silencing abilities of siRNAs that target the SMO gene, in each of which position 11 corresponded to the position of the point mutation, the base at the 5′-end of the guide strand was changed from cytosine to guanine, the group at the 2′-position of the pentose in each of ribonucleotides at positions 6-8 of the guide strand was replaced by OCH₃, and the base at position 6 of the guide strand was mismatched by changing it from guanine to cytosine, in an example of the present invention.

FIG. 19 shows graphs of the results on silencing abilities of siRNAs that target the AR gene, in each of which position 11 corresponded to the position of the point mutation, the base at the 5′-end of the guide strand was changed from guanine to uracil, the base at the 5′-end of the passenger strand was changed from uracil to guanine, the group at the 2′-position of the pentose in each of ribonucleotides at positions 6-8 of the guide strand was replaced by OCH₃, and the base at position 6 of the guide strand was mismatched by changing it from adenine to uracil, in an example of the present invention.

FIG. 20 shows graphs of the results on silencing abilities of siRNAs that target the DNM2 gene, in each of which position 11 corresponded to the position of the point mutation, the base at the 5′-end of the guide strand was changed from guanine to uracil, the base at the 5′-end of the passenger strand was changed from uracil to guanine, the group at the 2′-position of the pentose in each of ribonucleotides at positions 6-8 of the guide strand was replaced by OCH₃, and the base at position 6 of the guide strand was mismatched by changing it from cytosine to guanine, in an example of the present invention.

FIG. 21 shows graphs of the results on silencing abilities of siRNAs that target the KRT14 gene, in each of which position 11 corresponded to the position of the point mutation, the base at the 5′-end of the guide strand was changed from guanine to uracil, the group at the 2′-position of the pentose in each of ribonucleotides at positions 6-8 of the guide strand was replaced by OCH₃, and the base at position 6 of the guide strand was mismatched by changing it from guanine to cytosine, in an example of the present invention.

FIG. 22 shows graphs of the results on silencing abilities of a first siRNA that targets the IL4R gene, in which position 11 corresponded to the position of the point mutation, the base at the 5′-end of the passenger strand was changed from adenine to guanine, the group at the 2′-position of the pentose in each of ribonucleotides at positions 6-8 of the guide strand was replaced by OCH₃, and the base at position 6 of the guide strand was mismatched by changing it from adenine to uracil, in an example of the present invention.

FIG. 23 shows graphs of the results on silencing abilities of a second siRNA that targets the IL4R gene, in which position 11 corresponded to the position of the point mutation, the base at the 5′-end of the passenger strand was changed from uracil to guanine, the group at the 2′-position of the pentose in each of ribonucleotides at positions 6-8 of the guide strand was replaced by OCH₃, and the base at position 6 of the guide strand was mismatched by changing it from cytosine to guanine, in an example of the present invention.

FIG. 24 shows graphs of the results on silencing abilities of siRNAs that target the MAPT gene, in each of which position 11 corresponded to the position of the point mutation, the base at the 5′-end of the guide strand was changed from guanine to uracil, the base at the 5′-end of the passenger strand was changed from adenine to guanine, the group at the 2′-position of the pentose in each of ribonucleotides at positions 6-8 of the guide strand was replaced by OCH₃, and the base at position 6 of the guide strand was mismatched by changing it from cytosine to guanine, in an example of the present invention.

FIG. 25 shows graphs of the results on silencing abilities of siRNAs that target the MS4A2 gene, in each of which position 11 corresponded to the position of the point mutation, the base at the 5′-end of the guide strand was changed from adenine to uracil, the base at the 5′-end of the passenger strand was changed from cytosine to guanine, the group at the 2′-position of the pentose in each of ribonucleotides at positions 6-8 of the guide strand was replaced by OCH₃, and the base at position 6 of the guide strand was mismatched by changing it from adenine to uracil, in an example of the present invention.

FIG. 26 shows graphs of the results on silencing abilities of siRNAs that target the PABPNI gene, in each of which position 11 corresponded to the position of the point mutation, the base at the 5′-end of the guide strand was changed from guanine to uracil, the base at the 5′-end of the passenger strand was changed from adenine to guanine, the group at the 2′-position of the pentose in each of ribonucleotides at positions 6-8 of the guide strand was replaced by OCH₃, and the base at position 6 of the guide strand was mismatched by changing it from cytosine to guanine, in an example of the present invention.

FIG. 27 shows graphs of the results on silencing abilities of a first siRNA that targets the RHO gene, in which position 11 corresponded to the position of the point mutation, the base at the 5′-end of the guide strand was changed from guanine to uracil, the base at the 5′-end of the passenger strand was changed from adenine to guanine, the group at the 2′-position of the pentose in each of ribonucleotides at positions 6-8 of the guide strand was replaced by OCH₃, and the base at position 6 of the guide strand was mismatched by changing it from cytosine to guanine, in an example of the present invention.

FIG. 28 shows graphs of the results on silencing abilities of a second siRNA that targets the RHO gene, in which position 11 corresponded to the position of the point mutation, the base at the 5′-end of the passenger strand was changed from adenine to guanine, the group at the 2′-position of the pentose in each of ribonucleotides at positions 6-8 of the guide strand was replaced by OCH₃, and the base at position 6 of the guide strand was mismatched by changing it from cytosine to guanine, in an example of the present invention.

FIG. 29 shows graphs of the results on silencing abilities of siRNAs that target the SCNIA gene, in each of which position 11 corresponded to the position of the point mutation, the group at the 2′-position of the pentose in each of ribonucleotides at positions 6-8 of the guide strand was replaced by OCH₃, and the base at position 6 of the guide strand was mismatched by changing it from uracil to adenine, in an example of the present invention.

FIG. 30 shows graphs of the results on silencing abilities of siRNAs that target the APOB gene, in each of which position 11 corresponded to the position of the point mutation, the base at the 5′-end of the guide strand was changed from cytosine to uracil, the group at the 2′-position of the pentose in each of ribonucleotides at positions 6-8 of the guide strand was replaced by OCH₃, and the base at position 6 of the guide strand was mismatched by changing it from adenine to uracil, in an example of the present invention.

FIG. 31 shows graphs of the results on silencing abilities of siRNAs that target the F12 gene, in each of which position 11 corresponded to the position of the point mutation, the base at the 5′-end of the guide strand was changed from guanine to uracil, the base at the 5′-end of the passenger strand was changed from uracil to guanine, the group at the 2′-position of the pentose in each of ribonucleotides at positions 6-8 of the guide strand was replaced by OCH₃, and the base at position 6 of the guide strand was mismatched by changing it from guanine to cytosine, in an example of the present invention.

FIG. 32 shows graphs of the results on silencing abilities of siRNAs that target the CLCN7 gene, in each of which position 11 corresponded to the position of the point mutation, the base at the 5′-end of the guide strand was changed from adenine to uracil, the base at the 5′-end of the passenger strand was changed from uracil to guanine, the group at the 2′-position of the pentose in each of ribonucleotides at positions 6-8 of the guide strand was replaced by OCH₃, and the base at position 6 of the guide strand was mismatched by changing it from guanine to cytosine, in an example of the present invention.

FIG. 33 shows graphs of the results on silencing abilities of siRNAs that target the SCN8A gene, in each of which position 11 corresponded to the position of the point mutation, the base at the 5′-end of the guide strand was changed from cytosine to uracil, the base at the 5′-end of the passenger strand was changed from uracil to guanine, the group at the 2′-position of the pentose in each of ribonucleotides at positions 6-8 of the guide strand was replaced by OCH₃, and the base at position 6 of the guide strand was mismatched by changing it from cytosine to guanine, in an example of the present invention.

FIG. 34 shows graphs of the results on silencing abilities of siRNAs that target the PCSK9 gene, in each of which position 11 corresponded to the position of the point mutation, the base at the 5′-end of the guide strand was changed from guanine to uracil, the base at the 5′-end of the passenger strand was changed from cytosine to guanine, the group at the 2′-position of the pentose in each of ribonucleotides at positions 6-8 of the guide strand was replaced by OCH₃, and the base at position 6 of the guide strand was mismatched by changing it from guanine to cytosine, in an example of the present invention.

FIG. 35 shows graphs of the results on silencing abilities of siRNAs that target the KRT6A gene, in each of which position 11 corresponded to the position of the point mutation, the base at the 5′-end of the guide strand was changed from adenine to uracil, the base at the 5′-end of the passenger strand was changed from adenine to guanine, the group at the 2′-position of the pentose in each of ribonucleotides at positions 6-8 of the guide strand was replaced by OCH₃, and the base at position 6 of the guide strand was mismatched by changing it from guanine to cytosine, in an example of the present invention.

### EMBODIMENTS OF THE INVENTION

Objects, characteristics, advantages, and ideas of the present invention are apparent to a person skilled in the art from the description of the present specification, and the person skilled in the art can easily reproduce the present invention from the description of the present specification. Modes for carrying out the invention, specific examples thereof and so forth, which are described below, provide preferable embodiments of the present invention. They are described for the purpose of illustration or explanation, and thus the present invention is not limited thereto. It is apparent to a person skilled in the art that various alterations and modifications can be made on the basis of the description of the present specification within the spirit and the scope of the present invention disclosed in the present specification.

The nucleotide sequences herein are indicated such that their 5′- and 3′-ends are located on the left and right sides, respectively, unless otherwise specified.

### RNA Molecules

An embodiment of the present invention is RNA molecules for use in RNA interference to target a mutant allele of a gene, the mutant allele having a point mutation relative to its corresponding wild-type allele. Any gene may be targeted as long as the RNA molecules described herein can be designed for it: however, the target is preferably an oncogene that can transform normal cells by a point mutation, a causative gene responsible for a genetic disease developed by a point mutation, or a causative gene responsible for a disease with an SNP linked to a causative mutation in its coding region. It is preferable that the percentage of the SNP's linking to the causative mutation in a genetic pool is 50% or more. It is more preferable that the percentage is 60% or more, 70% or more, 80% or more, or 90% or more. It is even more preferable that the percentage is 95% or more, 99% or more, or 99.5% or more.

Examples of the oncogene include the ZMYM3, CTNNBI, SMARCA4, SMO, and AR genes. Examples of the causative gene responsible for a genetic disease include the DNM2, KRT14, IL4R, MAPT, MS4A2, PABPNI, SCNIA, APOB, F12, CLCN7, SCN8A, PCSK9, KRT6A, and RHO genes. Examples of the causative gene responsible for a disease with an SNP include the ATXN3 and HTT genes. Diseases that would be caused by these mutations are listed in Table 1.

The RNA molecules herein may consist of any number of nucleotides, and the number may be 13 or more and 100 or less, 13 or more and 50 or less, 13 or more and 28 or less, 15 or more and 25 or less, or 17 or more and 21 or less. More preferably, the number is 19 or more and 21 or less. One or more ribonucleotides may be each replaced by a deoxyribonucleotide, an artificial nucleic acid, or a nucleic acid analog such as inosine or morpholino. Such RNA molecules are herein called “chimeric NA molecules,” and the RNA molecules are described as including chimeric NA molecules in the present disclosure.

In the RNA molecules, the base at position 5 or 6, counted from the base at the 5′-end of a nucleotide sequence complementary to that of a mutant allele, is mismatched with the base of the mutant allele. The RNA molecules have a nucleotide sequence complementary to that of the coding region of the mutant allele in the rest of the nucleotide sequence. Furthermore, the RNA molecules may have a sequence other than the nucleotide sequence complementary to that of the coding region of the mutant allele, such as a sequence complementary to the complementary nucleotide sequence, with which the molecules may be self-annealed to function as siRNA. An example of such a single-stranded RNA is Bonac nucleic acid. Alternatively, 1-3 nucleotide(s) may be added to its 3′-end, whose nucleotide sequences are not limited. If the base at the 5′-end of the complementary nucleotide sequence is not adenine or uracil, it may be replaced by adenine, uracil or thymine. If the base at the 3′-end of the complementary nucleotide sequence is not cytosine or guanine, it may be replaced by cytosine or guanine. These modifications enhance the gene expression suppression ability of the RNA molecules when they work as siRNA's guide strands. The RNA molecules may also contain chemical substances in addition to nucleic acids for delivery, to increase membrane permeability, or improve blood retention. For example, the RNA molecules may be conjugated to Ga1NAc or PEG. The RNA molecules may consist of a sequence other than the nucleotide sequence completely complementary to that of the coding region of the mutant allele except for the base at position 5 or 6. Notably, the RNA molecules have a nucleotide sequence complementary to that of the mutant allele except for the base at position 5 or 6, and their sequence complementarity is preferably 90% or more, more preferably 95% or more, yet more preferably 98% or more, and most preferably 100%. The base at position 5 or 6 may be any base, provided it is mismatched with that of the mutant allele. The base may be A, U, C, G, T, I, or any other artificial nucleic acid or nucleic acid analog as long as it differs from that in the corresponding position of the mutant allele.

Moreover, in the RNA molecules, the base at position 10 or 11, counted from the base at the 5′-end of the nucleotide sequence complementary to that of the mutant allele, is at the position of the point mutation and is identical to the base in the corresponding position of the mutant allele. In the case that the mutated base in the mutant allele is adenine, cytosine, guanine, or thymine, the base at position 10 or 11 of the RNA molecules is adenine, cytosine, guanine, or uracil (or thymine), respectively.

In the RNA molecules, the group at the 2′-position of the pentose in each of their nucleotides at positions 6-8 or positions 7 and 8, counted from the base at the 5′-end of the nucleotide sequence complementary to that of the mutant allele, is independently modified with (i.e., replaced by) OCH₃, halogen, or LNA. For example, RNA in which the group at the 2′-position of the pentose is replaced by —OCH₃ (hereinafter referred to as a “2′-O-methyl RNA”) has a structure represented by the following general formula:

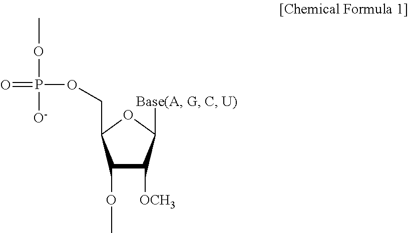

Any halogen may be used, but fluorine is preferred because of its small molecular size. For nucleotides at positions other than those mentioned, some or all of them may be modified; however, it is preferable that none of them is modified. Modifications of nucleotides are not particularly limited and exemplified that the group at the 2′-position of the pentose in the nucleotides is replaced by a group selected from the group consisting of H, OR, R, halogen, SH, SR, NH₂, NHR, NR₂, CN, COOR, and LNA, in which R is C₁-C₆ alkyl, alkenyl, alkynyl, or aryl; and halogen is F, Cl, Br, or I.

The IC50 of the RNA molecules for the target is preferably 1 nM or less, more preferably 500 pM or less, and yet more preferably 200 pM or less.

When the RNA molecules are used for RNA interference as a single strand, it is preferable that their 5′-end is phosphorylated or can be phosphorylated in situ or in vivo.

A method of designing the RNA molecules includes the following steps.

First, the step is performed in which a nucleotide sequence of a certain length containing a sequence complementary to that of a mutant allele is designed such that a mutated base in the mutant allele is placed at tenth or eleventh position, counted from the base at the 5′-end. The next step is to place a mismatched base at position 5 or 6, counted from the base at the 5′-end. Then, the group at the 2′-position of the pentose in each of nucleotides at positions 6-8 or positions 7 and 8, counted from the base at the 5′-end, is independently modified with OCH₃, halogen, or LNA. In the case that the base at the 5′-end of the complementary nucleotide sequence is not adenine or uracil, the step of replacing it by adenine or uracil or thymine may be performed. Likewise, in the case that the base at the 3′-end of the complementary nucleotide sequence is not cytosine or guanine, the step of replacing it by cytosine or guanine may be performed. Finally, 1-3 base(s) may be added to the 3′-end. In this way, a nucleotide sequence can be designed. A program for causing a computer to perform this design method may be made, and the program may be stored in a computer-readable recording medium. Nucleotides with a sequence designed in this manner can be chemically synthesized according to a routine method.

### Double-Stranded RNA Molecules

An embodiment of the present invention is double-stranded RNA molecules in which one of the aforementioned RNA molecules (hereinafter referred to as a “first RNA molecule”) serves as a guide strand, and a second RNA molecule with a sequence complementary to that of the first RNA molecule serves as a passenger strand. The second RNA molecule has a sequence complementary to that of the first RNA molecule and forms a duplex with the first RNA molecule under physiological conditions. Their sequence complementarity is preferably 90% or more, more preferably 95% or more, yet more preferably 98% or more, and most preferably 100%.

The passenger strand may have any length and may be considerably shorter than the first RNA molecule. For example, the length of the passenger strand may be equal to or less than half the length of the first RNA molecule. It is, however, preferable that they have the same length. When the passenger strand is shorter than the first RNA molecule, the latter has a single-stranded portion. This portion may be left single-stranded, or alternatively, a third RNA molecule complementary to the first RNA molecule may be annealed to the single-stranded portion. In the case that the second and third RNA molecules occupy the entire length of the first RNA molecule, the resulting double strand is identical to the one with a nick present on a single passenger strand to divide it into two.

The double-stranded RNA molecules may have two blunt ends, or alternatively, they may have an overhang at the 3′-end of either or both first and second RNA molecules serving as the guide and passenger strands, respectively. The overhang(s) may have any number of nucleotides but is/are preferably of 1-3 nucleotides long.

The double-stranded RNA molecule may be a double-stranded chimeric NA molecule in which 1-3, 4-6, 7-9, 10-12, 13-15, 16-18, 19-21, 22-24, or 25 or more ribonucleotides, or all ribonucleotides are each replaced by a deoxyribonucleotide, an artificial nucleic acid such as morpholine, or a nucleic acid analog such as glycol nucleic acid. The replaced positions are not limited.

The nucleotides in the passenger strand may be modified; however, it is preferable that they are not modified. Modifications of nucleotides are not particularly limited, and, for example, the group at the 2′-position of the pentose in the nucleotides may be replaced by a group selected from the group consisting of H, OR, R, halogen, SH, SR₁, NH₂, NHR, NR₂, CN, COOR, and LNA, in which R is C₁-C₆ alkyl, alkenyl, alkynyl, or aryl; and halogen is F, Cl, Br, or I. Passenger strands can also be easily designed and easily produced using known techniques. A guide strand and a passenger strand may be linked to each other by a linker. The linker may be formed of any material, and examples include peptides and PEG.

### RNA Interference

An embodiment of the present invention is a method for performing RNA interference by which a mutant allele with a point mutation is targeted in cells containing the wild-type allele of a gene of interest and a mutant allele of the gene.

This method includes the step of introducing the first RNA molecule that may include a chimeric NA molecule, or one of the aforementioned double-stranded RNA molecules that may include a double-stranded chimeric NA molecule, into the cells containing the wild-type allele and the mutant allele.

RNA interference can be readily performed using known techniques. The expression of a target can be reduced by introducing the first RNA molecule or the double-stranded RNA molecule into, for example, culture cells or a human or non-human individual organism expressing the target gene.

The use of the aforementioned first RNA molecules or double-stranded RNA molecules in RNA interference makes it possible to primarily suppress the expression of the mutant allele of the gene of interest, substantially not suppressing the expression of the wild-type allele. Here, the expression of the wild-type allele may be suppressed up to the level at which the wild-type allele is functional and a normal phenotype is exhibited. The expression of the mutant allele should be inhibited at least to the level at which the mutant allele is not functional and the abnormal phenotype is not exhibited. This enables, for example, cells to become functional normally without developing the phenotype due to a mutation even when the mutant allele carries a dominant mutation.

### Agents

An embodiment of the present invention is a therapeutic agent for a patient with a disease or a prophylactic agent for a carrier of the disease, the patient or the carrier having wild-type and mutant alleles of a causative gene for the disease, the mutant allele of the causative gene having a point mutation and being responsible for the disease, wherein the therapeutic or prophylactic agent includes, as an active ingredient, any of the aforementioned RNA molecules including any one of the aforementioned chimeric NA molecules or any one of double-stranded RNA molecules including any one of the aforementioned double-stranded chimeric NA molecules. Carriers as used herein refer to individuals with a mutant allele of a causative gene for a disease who have not developed it and may develop it in the future. Prophylactic agents for carriers help prevent them from developing the disease due to the mutant allele of the causative gene.

Here, the cause of the disease may not be the point mutation but rather a different mutation and a certain percentage of patients or carriers of the disease have the point mutation. In the latter cases, the point mutation is preferably associated with the causative mutation. The aforementioned percentage is preferably 50% or more: more preferably, 60% or more, 70% or more, 80% or more, or 90% or more: and yet more preferably, 95% or more, 99% or more, or 99.5% or more, although not specifically limited. If the percentage is low, the patient or carrier may be examined to determine whether they have the point mutation before administering the agent. In these cases, healthy persons other than the patient or carrier preferably do not have the point mutation.

Examples of the former cases are genetic diseases and tumors that are caused by a point mutation. The genetic diseases are not limited as long as their development is caused by a point mutation, among which some examples are given in Table 1. Likewise, the tumors are not limited as long as they are caused by a point mutation in an oncogene, among which some examples are given in Table 1.

Examples of the latter include triplet repeat diseases. Triplet repeat diseases are known to be caused by 5-40 repeats and 36-3000 repeats of a triplet sequence such as CAG in healthy individuals and patients, respectively. For example, in the ATXN3 mutant gene, which is a causative gene for Machado-Joseph disease, an SNP in which G immediately after a CAG repeat is mutated to C can be found. This mutation could be the target of the siRNA of the present disclosure. The triplet repeat diseases are not limited, among which some examples are given in Table 1.

Any method can be used for the administration of the agents disclosed herein; however, injection is preferable, and intravenous injection is more preferable. In such cases, in addition to the active ingredient, other ingredients such as pH adjusters, buffers, stabilizers, tonicity adjusting agents, or local anesthetics may be added to the therapeutic agents.

The dosage of the therapeutic agents is not limited and is selected as appropriate based on, for example, the efficacy of the ingredients contained, the mode of administration, the route of administration, the type of the disease, attributes of a subject (e.g., weight, age, medical conditions, and history of use of other medicaments), and the discretion of a physician in charge.

### Selection Methods

An embodiment of the present invention is a method for selecting RNA molecules, chimeric NA molecules, double-stranded RNA molecules, or double-stranded chimeric NA molecules for use in RNA interference to silence a target, the selection method including the steps of evaluating a gene-specific silencing ability of a plurality of the aforementioned RNA molecules, chimeric NA molecules, double-stranded RNA molecules, or double-stranded chimeric NA molecules by performing RNA interference in vitro using them; and selecting RNA molecules, chimeric NA molecules, double-stranded RNA molecules, or double-stranded chimeric NA molecules having at least a certain level of the gene-specific silencing ability.

In performing RNA interference in vitro, a wild-type allele and a mutant allele with a point mutation, of a gene are used as targets, and a molecule is selected which does not suppress the expression of the wild-type allele to a given level but suppresses the expression of the mutant allele to a level equal to or lower than a given level. By using this procedure, one or more molecules that suppress the expression of the mutant allele but do not suppress the expression of the wild-type allele can be obtained. Here, the given level may be any level, but is preferably 50%, more preferably 70%, and even more preferably 90%.

Methods for the assay using RNA interference in vitro are common knowledge in the art, and the selection of genes, selection of cells, and introduction of RNA molecules into cells, etc. are obvious to those skilled in the art.

### EXAMPLES

### Method

HeLa cells cultured in DMEM supplemented with 10% FBS were seeded at 1×10⁵ cells/mL on 24-well plates and co-transfected with each double-stranded siRNA with 100 ng of a reporter and 100 ng of plasmid (pGL3) as an internal standard, using 2 μL of lipofectamine 2000. The concentrations of the double-stranded siRNAs are shown in the figures. siGY441 was introduced as a control for siRNA. The cells were harvested after 24 hours, and firefly and Renilla luciferase activities were measured using a dual-luciferase reporter assay system (Promega). Renilla luciferase activity was normalized from the firefly luciferase activity. The results obtained for the double-stranded siRNAs are presented in graphs, relative to those for siGY441, which were set to 100%.

### Example 1

In this example, the K-ras gene was used as a target gene to be silenced.

### Example 1-1

This example shows that, by matching position 10 or 11 of each siRNA with the position of the point mutation in the A-mutant allele of the K-ras gene (c. 35G>A) (hereinafter, referred to as the “A-mutant allele”), the RNA molecules exhibit a higher specificity for silencing abilities to the A-mutant allele of the K-ras gene (35G>A) than to the wild-type allele of the K-ras gene (hereinafter, referred to as a “wild-type allele”).

First, as reporters for examining gene silencing effects, DNAs with the same nucleotide sequences as the wild-type allele of the K-ras gene (wt) and the A-mutant allele of the K-ras gene (c. 35G>A) were chemically synthesized and inserted into the 3′-UTR of the luciferase gene in an expression vector (psiCHECK) to construct wild-type and A-mutant K reporters, respectively. The sequences of the segments incorporated into the vectors are indicated below.

| | |
|---|---|
| Wild-type K reporter: | |
| 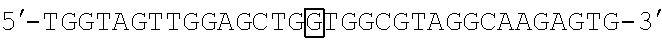 | (SEQ ID NO. 4) |
| 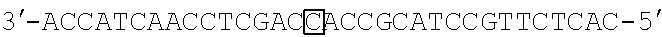 | (SEQ ID NO. 5) |
| A-mutant K reporter: | |
| 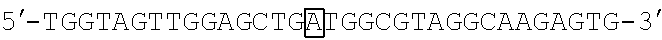 | (SEQ ID NO. 6) |
| 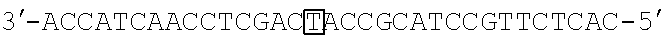 | (SEQ ID NO. 7) |

Next, double-stranded RNAs with the following sequences were chemically synthesized for siRNAs. The positions 9, 10, and 11 in siRNAs, K(35)9A, K(35)10A, and K(35)11A, respectively, correspond to the position of the point mutation in the A-mutant allele of the K-ras gene (c. 35G>A). In the following sequences, base pairs in the position corresponding to the position of the point mutation are enclosed in rectangles.

| | |
|---|---|
| K(35)9A: | |
| 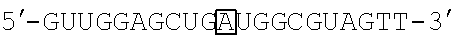 | (SEQ ID NO. 8) |
| 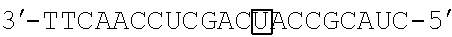 | (SEQ ID NO. 9) |
| K(35)10A: | |
| 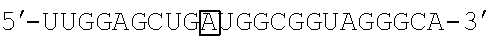 | (SEQ ID NO. 10) |
| 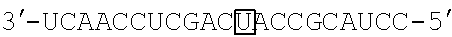 | (SEQ ID NO. 11) |
| K(35)11A: | |
| 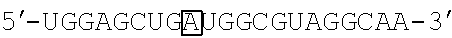 | (SEQ ID NO. 12) |
| 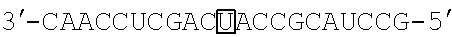 | (SEQ ID NO. 13) |

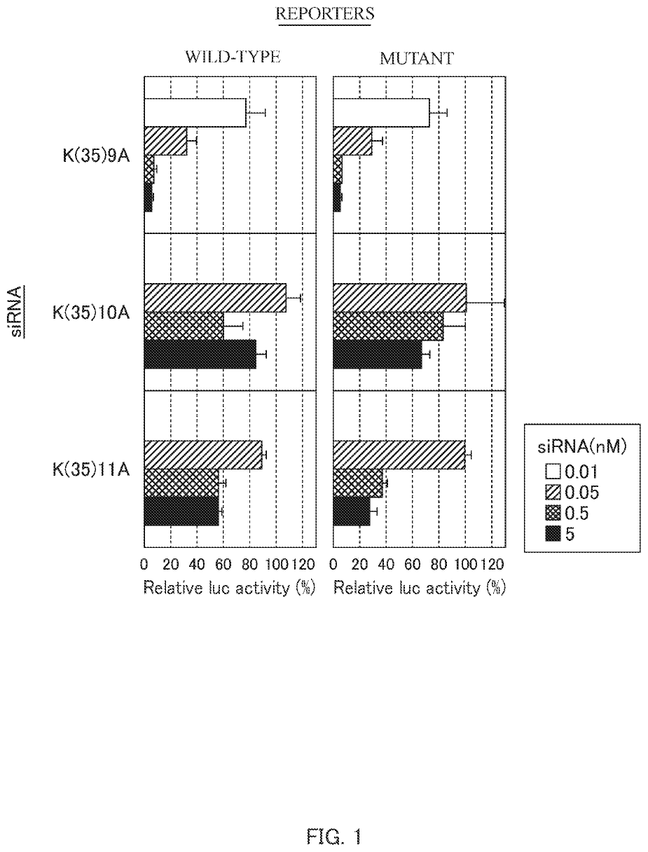

FIG. 1 shows gene silencing effects of the siRNAs.

K(35)9A had a strong silencing effect on both A-mutant and wild-type alleles. K(35)10A and K(35)11A strongly suppressed the expression of the A-mutant allele more than that of the wild-type allele although their silencing effects were slightly reduced.

### Example 1-2

This example shows that the silencing abilities of the RNA molecule to the A-mutant allele become stronger and its specificities become much higher by, in addition to matching position 11 of an siRNA with the position of the point mutation in the A-mutant allele, changing the base at the 5′-end of the siRNA's guide strand from guanine to uracil and changing the base at the 5′-end of the passenger strand from uracil to guanine.

The wild-type and A-mutant K reporters were used as reporters for examining gene silencing effects. A double-stranded RNA with the following sequences was chemically synthesized for an siRNA, and K(35)11A was used as a control. In the following sequences, a base pair in the position corresponding to the position of the point mutation, and the pairs of the modified bases at the 5′-ends of the guide and passenger strands are enclosed in rectangles.

| | |
|---|---|
| K(35)11Arev: | |
| 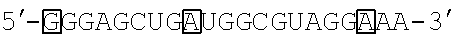 | (SEQ ID NO. 14) |
| 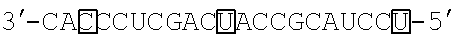 | (SEQ ID NO. 15) |

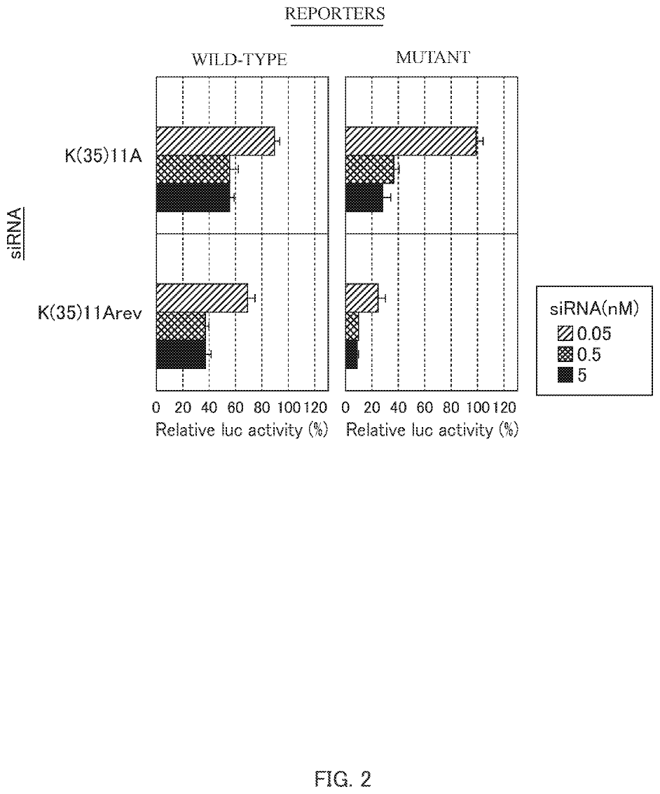

FIG. 2 shows gene silencing effects of the siRNAs.

K(35) 11A strongly suppressed the expression of the A-mutant allele more than that of the wild-type allele, whereas K(35)11Arev exerted a stronger silencing effect on both, with a stronger suppression of the expression of the A-mutant allele than that of the wild-type allele.

### Example 1-3

This example shows that silencing abilities of the RNA molecules to the A-mutant allele become stronger and their specificities become much higher by, in addition to matching position 11 of each siRNA with the position of the point mutation in the A-mutant allele, changing the base at the 5′-end of the siRNA's guide strand from guanine to uracil, and changing the base at the 5′-end of the passenger strand from uracil to guanine, replacing the group at 2′-position of the pentose in each of ribonucleotides at positions 6-8 of the guide strand by OCH₃.

The wild-type and A-mutant K reporters were used as reporters for examining gene silencing effects. Double-stranded RNAs with the following sequences were chemically synthesized for siRNAs, and K(35)11 Arev was used as a control. In the following sequences, the base pairs in the position corresponding to the position of the point mutation, and the pairs of the modified bases at the 5′-ends of the guide and passenger strands are enclosed in rectangles. The nucleotides in which the group at the 2′-position of the pentose was replaced by OCH₃ are hatched.

| | |
|---|---|
| K(35)11ArevOM(2-5): | |
| 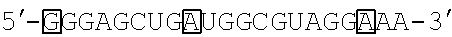 | (SEQ ID NO. 16) |
| 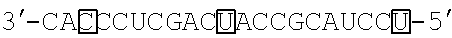 | (SEQ ID NO. 17) |
| K(35)11ArevOM(6-8): | |
| 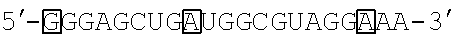 | (SEQ ID NO. 18) |
| 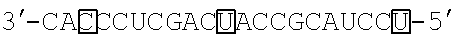 | (SEQ ID NO. 19) |

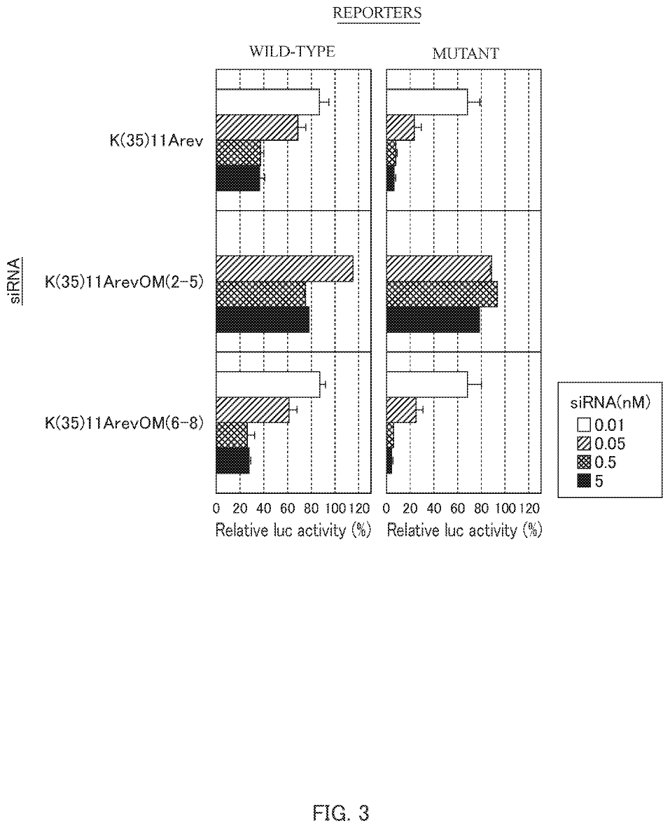

FIG. 3 shows gene silencing effects of the siRNAs.

K(35) 11 Arev strongly suppressed the expression of the A-mutant allele more than that of the wild-type allele, whereas K(35) 11ArevOM(6-8) exerted a stronger silencing effect on both, with a stronger suppression of the expression of the A-mutant allele than that of the wild-type allele. Another control, K(35)11 ArevOM(2-5) in which the group at the 2′-position of the pentose in each of ribonucleotides at positions 2-5 of the guide strand was replaced by OCH₃ exerted considerably weak silencing effect on both.

### Example 1-4

This example shows that silencing abilities of the RNA molecules to the wild-type allele become weaker and, as a result, their specificities for the A-mutant allele become much higher by, in addition to matching position 11 of each siRNA with the position of the point mutation in the A-mutant allele, changing the base at the 5′-end of the siRNA's guide strand from guanine to uracil, and changing the base at the 5′-end of the passenger strand from uracil to guanine, mismatching the base at position 5 or 6 of the guide strand with that of the A-mutant allele,.

The wild-type and A-mutant K reporters were used as reporters for examining gene silencing effects. Double-stranded RNAs with the following sequences with a mismatched base at one of positions 3-7 based on K(35)11Arev were chemically synthesized for siRNAs. K(35)11Arev was used as a control. In the following sequences, the base pairs in the position corresponding to the position of the point mutation, the pairs of the modified bases at the 5′-ends of the guide and passenger strands, and base pairs with the mismatched base are enclosed in rectangles.

| | |
|---|---|
| K(35)11ArevM3: | |
| 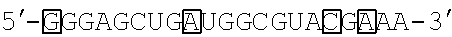 | (SEQ ID NO. 20) |
| 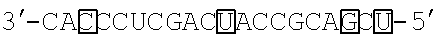 | (SEQ ID NO. 21) |
| K(35)11ArevM4: | |
| 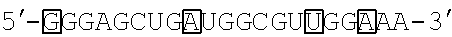 | (SEQ ID NO. 22) |
| 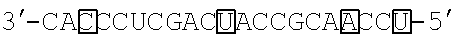 | (SEQ ID NO. 23) |
| K(35)11ArevM5: | |
| 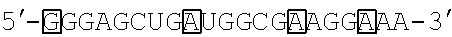 | (SEQ ID NO. 24) |
| 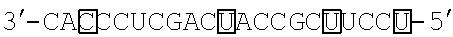 | (SEQ ID NO. 25) |
| K(35)11ArevM6: | |
| 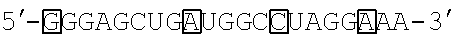 | (SEQ ID NO. 26) |
| 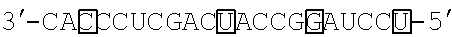 | (SEQ ID NO. 27) |
| K(35)11ArevM7: | |
| 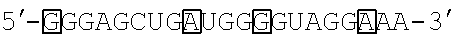 | (SEQ ID NO. 28) |
| 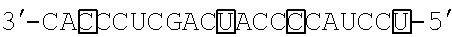 | (SEQ ID NO. 29) |

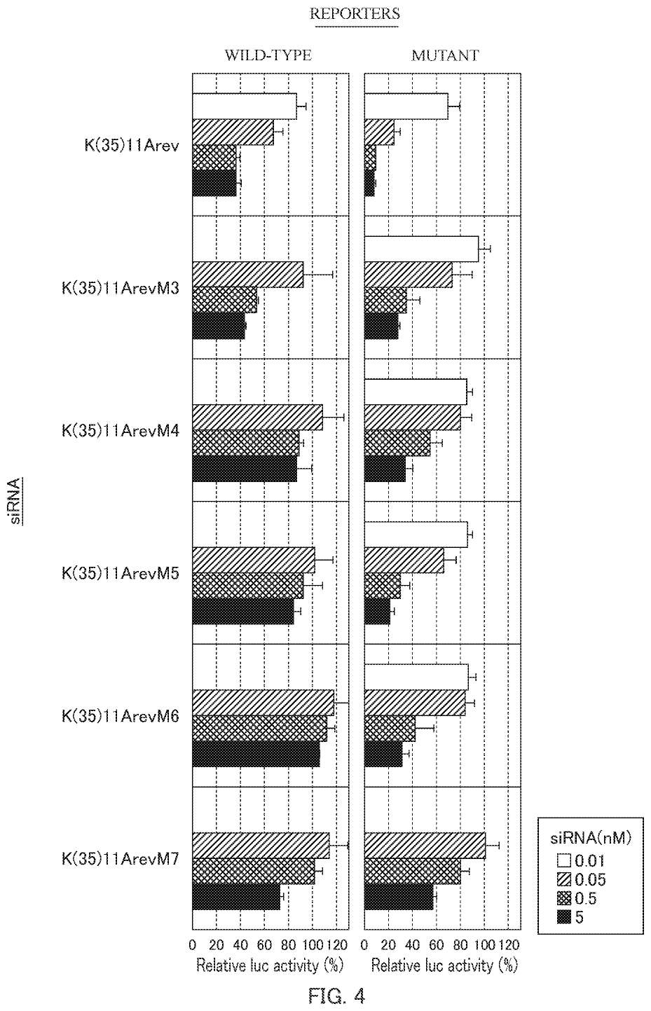

FIG. 4 shows gene silencing effects of the siRNAs.

K(35) 11 Arev strongly suppressed the expression of the A-mutant allele more than that of the wild-type allele, whereas RNA molecules K(35) 11 ArevM5 and K(35)11 ArevM6 exhibited significantly weak silencing abilities to the wild-type allele and, as a result, much higher specificities for the A-mutant allele.

### Example 1-5

This example shows that specificities of the RNA molecules for the A-mutant allele become much higher by, in addition to matching position 11 of each siRNA with the position of the point mutation in the A-mutant allele, changing the base at the 5′-end of the siRNA's guide strand from guanine to uracil, and changing the base at the 5′-end of the passenger strand from uracil to guanine, replacing the group at the 2′-position of the pentose in each of ribonucleotides at positions 6-8 of the guide strand by OCH₃, and mismatching the base at position 5 or 6 of the guide strand with that of the A-mutant allele.

The wild-type and A-mutant K reporters were used as reporters for examining gene silencing effects. Double-stranded RNAs with the following sequences with a mismatched base at one of positions 3-7 based on K(35)11Arev were chemically synthesized for siRNAs. K(35)11Arev was used as a control. In the following sequences, the base pairs in the position corresponding to the position of the point mutation, the pairs of the modified bases at the 5′-ends of the guide and passenger strands, and base pairs with the mismatched base are enclosed in rectangles. The nucleotides in which the group at the 2′-position of the pentose was replaced by OCH₃ are hatched.

| | |
|---|---|
| K(35)11ArevOM(6-8)M3: | |
| 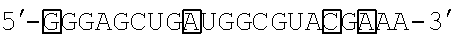 | (SEQ ID NO. 30) |
| 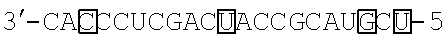 | (SEQ ID NO. 31) |
| K(35)11ArevOM(6-8)M4: | |
| 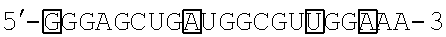 | (SEQ ID NO. 32) |
| 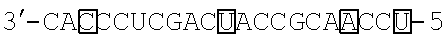 | (SEQ ID NO. 33) |
| K(35)11ArevOM(6-8)M5: | |
| 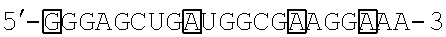 | (SEQ ID NO. 34) |
| 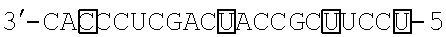 | (SEQ ID NO. 35) |
| K(35)11ArevOM(6-8)M6: | |
| 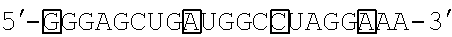 | (SEQ ID NO. 36) |
| 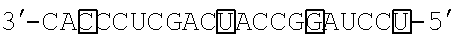 | (SEQ ID NO. 37) |
| K(35)11ArevOM(6-8)M7: | |
| 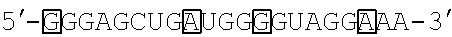 | (SEQ ID NO. 38) |
| 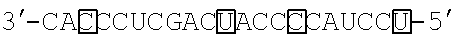 | (SEQ ID NO. 39) |

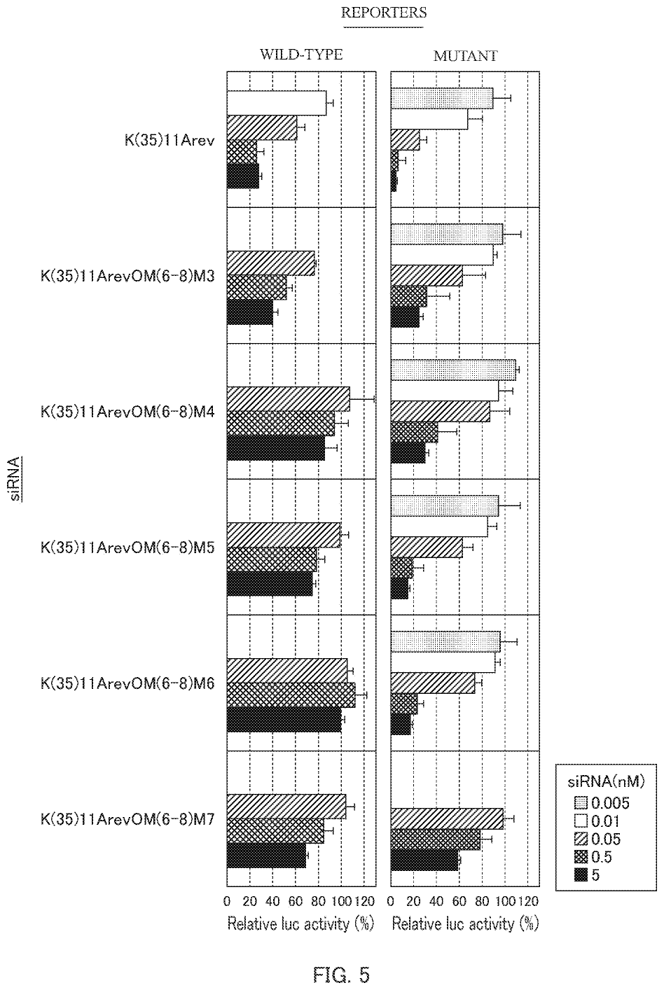

FIG. 5 shows gene silencing effects of the siRNAs. K(35)11ArevOM(6-8)M5 and K(35)11ArevOM(6-8)M6 exhibited very weak silencing abilities to the wild-type allele and, as a result, their specificities for the A-mutant allele became much higher.

### Example 1-6

This example shows that siRNAs specific for the A-mutant allele exhibit weak silencing abilities not only to the wild-type allele, but also to the T-mutant allele of the K-ras gene (c. 35G>T) and the C-mutant allele of the K-ras gene (c. 35 G>C).

K-ras reporters indicated below, i.e., the wild-type K reporter, the A-mutant K reporter, a T-mutant reporter for the K-ras gene (c. 35G>T) (hereinafter, referred to as the “T-mutant K reporter”), and a C-mutant reporter for the K-ras gene (c. 35 G>C) (hereinafter, referred to as the “C-mutant K reporter”), were used as reporters for examining gene silencing effects. K(35)11ArevOM(6-8)M5 and K(35)11 ArevOM(6-8)M6 were used as siRNAs, and K(35)11Arev was used as a control. In the following sequences, the base pairs in the position corresponding to the position of the point mutation are enclosed in rectangles.

| | |
|---|---|
| T-mutant K reporter: | |
| 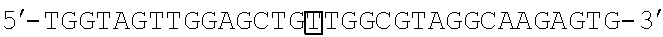 | (SEQ ID NO. 40) |
| 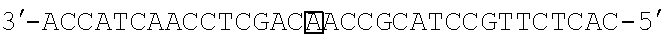 | (SEQ ID NO. 41) |
| C-mutant K reporter: | |
| 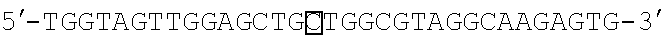 | (SEQ ID NO. 42) |
| 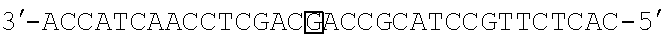 | (SEQ ID NO. 43) |

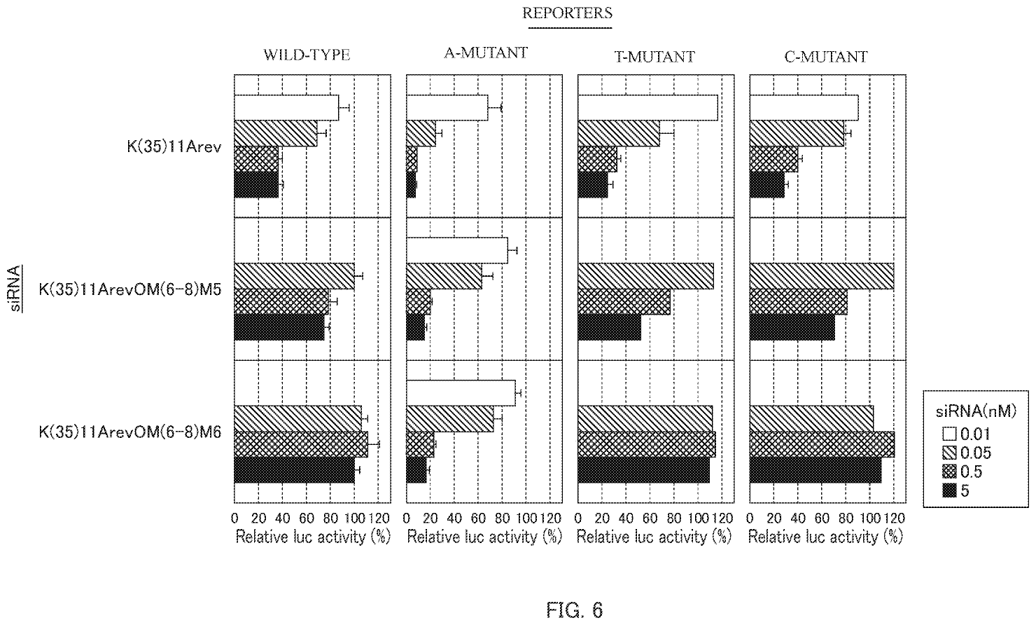

FIG. 6 shows gene silencing effects on the reporters.

All siRNAs had the strongest silencing effect on the A-mutant K reporter; especially K(35) 11 ArevOM(6-8)M5 and K(35) 11ArevOM(6-8)M6 had weak silencing effects on the T-mutant and C-mutant K reporters.

### Example 1-7

This example shows that siRNAs specific for the T-mutant allele exhibit weak silencing abilities to the A-mutant and C-mutant alleles in addition to the wild-type allele.

K-ras reporters, i.e., the wild-type, A-mutant, T-mutant, and C-mutant K reporters, were used as reporters for examining gene silencing effects. K(35)11TrevOM(6-8)M5 and K(35)11TrevOM(6-8)M6 were used as siRNAs, and K(35) 11 Trev was used as a control. In the following sequences, the base pairs in the position corresponding to the position of the point mutation, the pairs of the modified bases at the 5′-ends of the guide and passenger strands, and the base pairs with the mismatched base are enclosed in rectangles. The nucleotides in which the group at 2′-position of the pentose was replaced by OCH₃ are hatched.

| | |
|---|---|
| K(35)11Trev: | |
| 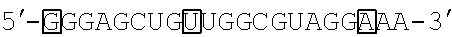 | (SEQ ID NO. 44) |
| 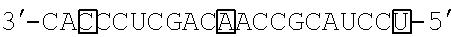 | (SEQ ID NO. 45) |
| K(35)11TrevOM(6-8)M5: | |
| 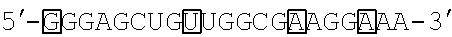 | (SEQ ID NO. 46) |
| 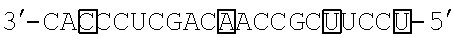 | (SEQ ID NO. 47) |
| K(35)11TrevOM(6-8)M6: | |
| 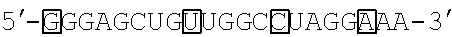 | (SEQ ID NO. 48) |
| 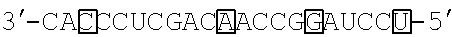 | (SEQ ID NO. 49) |

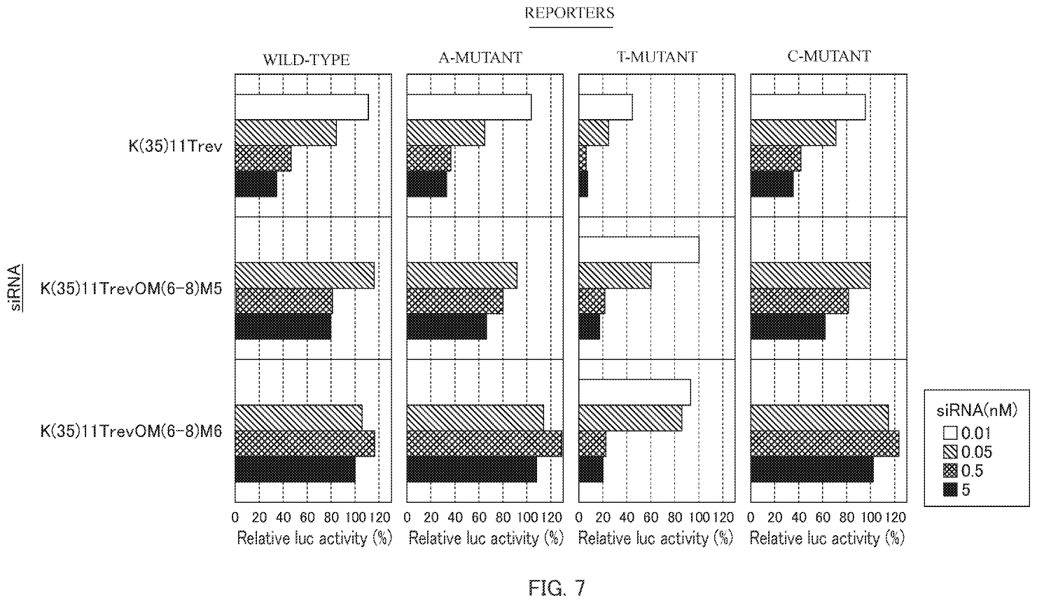

FIG. 7 shows gene silencing effects on the reporters.

All siRNAs had the strongest silencing effect on the T-mutant K reporter; especially K(35) 11TrevOM(6-8)M5 and K(35)11TrevOM(6-8)M6 had weak silencing effects on the T-mutant and C-mutant K reporters.

### Example 1-8

This example shows that siRNAs specific for the C-mutant allele exhibit weak silencing abilities to the A-mutant and T-mutant alleles in addition to the wild-type allele.

K-ras reporters, i.e., the wild-type, A-mutant, T-mutant, and C-mutant K reporters were used as reporters for examining gene silencing effects. K(35)11CrevOM(6-8)M5 and K(35)11CrevOM(6-8)M6 were used as siRNAs, and K(35)11 Crev was used as a control. In the following sequences, the base pairs in the position corresponding to the position of the point mutation, the pairs of the modified bases at the 5′-ends of the guide and passenger strands, and the base pairs with the mismatched base are enclosed in rectangles. The nucleotides in which the group at the 2′-position of the pentose was replaced by OCH₃ are hatched.

| | |
|---|---|
| K(35)11Crev: | |
| 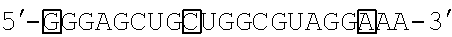 | (SEQ ID NO. 50) |
| 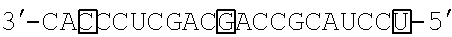 | (SEQ ID NO. 51) |
| K(35)11CrevOM(6-8)M5: | |
| 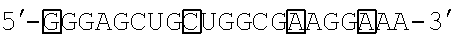 | (SEQ ID NO. 52) |
| 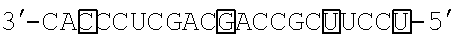 | (SEQ ID NO. 53) |
| K(35)11CrevOM(6-8)M6: | |
| 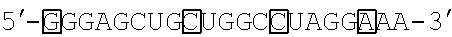 | (SEQ ID NO. 54) |
| 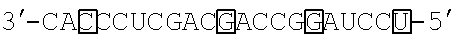 | (SEQ ID NO. 55) |

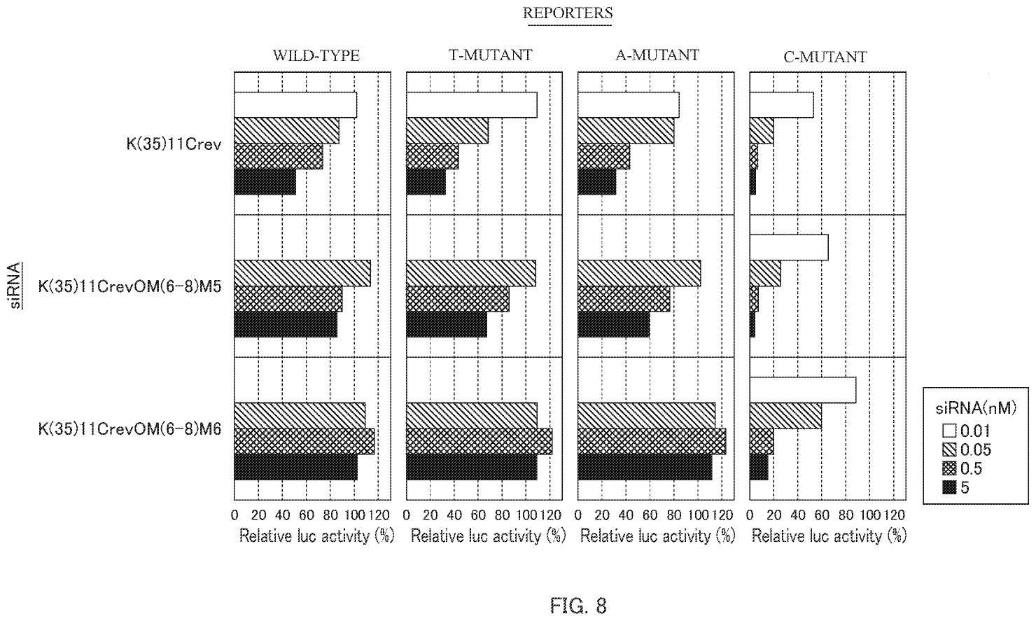

FIG. 8 shows gene silencing effects on the reporters.

All siRNAs had the strongest silencing effect on the C-mutant K reporter; especially K(35) 11CrevOM(6-8) M5 and K(35)11CrevOM(6-8)M6 had weak silencing effect on the A-mutant and T-mutant K reporters.

### Example 2

In this example, the N-ras gene was chosen as a target gene to be silenced.

### Example 2-1

This example, targeting the point mutation in nt 35 of N-ras cDNA, shows that siRNAs suppress the expression of the A-mutant N35 allele more specifically than that of the wild-type allele of the N-ras (wt) gene (hereinafter, referred to as the “wild-type N allele”) by matching position 11 of each siRNA with the position of the point mutation in the A-mutant allele of the N-ras gene (c. 35G>A) (hereinafter, referred to as the “A-mutant N35 allele”), changing the base at the 5′-end of the guide strand of the siRNA from cytosine to uracil, changing the base at the 5′-end of the passenger strand from adenine to guanine, replacing the group at the 2′-position of the pentose in each of ribonucleotides at positions 6-8 of the guide strand by OCH₃, and mismatching the base at position 5 of the guide strand with that of the A-mutant N35 allele.

First, as reporters for examining gene silencing effects, DNAs with the same nucleotide sequences as the wild-type N allele and the A-mutant N35 allele were inserted into the 3′-UTR of the luciferase gene in an expression vector (psiCHECK) to construct wild-type N35 and A-mutant N reporters, respectively. The sequences of the segments chemically synthesized and incorporated into the vectors are indicated below. In the following sequences, the base pairs in the position corresponding to the position of the point mutation are enclosed in rectangles.

| | |
|---|---|
| Wild-type N35 reporter: | |
| 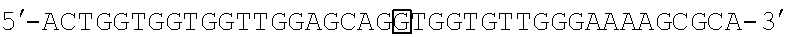 | (SEQ ID NO. 56) |
| 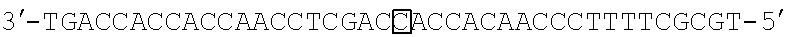 | (SEQ ID NO. 57) |
| A-mutant N35 reporter: | |
| 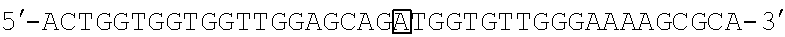 | (SEQ ID NO. 58) |
| 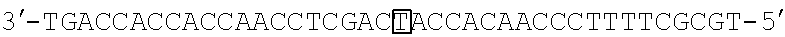 | (SEQ ID NO. 59) |

Double-stranded RNAs with the following sequences were chemically synthesized for siRNAs. N(35)11G has a sequence complementary to that of the wild-type N allele. N(35)11A is an siRNA in which position 11 corresponded to the position of the point mutation in the A-mutant N35 allele. N(35) 11 ArevOM(6-8)M5 is an siRNA in which position 11 corresponded to the position of the point mutation in the A-mutant N35 allele, the base at the 5′-end of the guide strand was changed from cytosine to uracil, the base at the 5′-end of the passenger strand was changed from uracil to cytosine, the group at the 2′-position of the pentose in each of ribonucleotides at positions 6-8 of the guide strand was replaced by OCH₃, and the base at position 5 of the siRNA's guide strand was mismatched with that of the A-mutant N35 allele. In the following sequences, the base pairs in the position corresponding to the position of the point mutation, the pairs of the modified bases at the 5′-ends of the guide and passenger strands, and the base pairs with the mismatched base are enclosed in rectangles. The nucleotides in which the group at the 2′-position of the pentose was replaced by OCH₃ are hatched.

| | |
|---|---|
| N(35)11G: | |
| 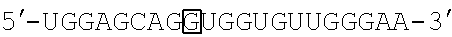 | (SEQ ID NO. 60) |
| 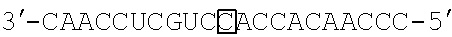 | (SEQ ID NO. 61) |
| N(35)11A: | |
| 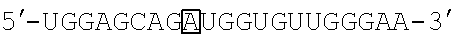 | (SEQ ID NO. 62) |
| 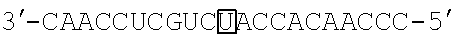 | (SEQ ID NO. 63) |
| N(35)11ArevOM(6-8)M5: | |
| 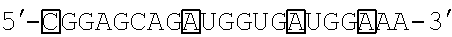 | (SEQ ID NO. 64) |
| 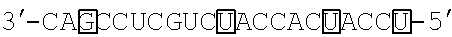 | (SEQ ID NO. 65) |

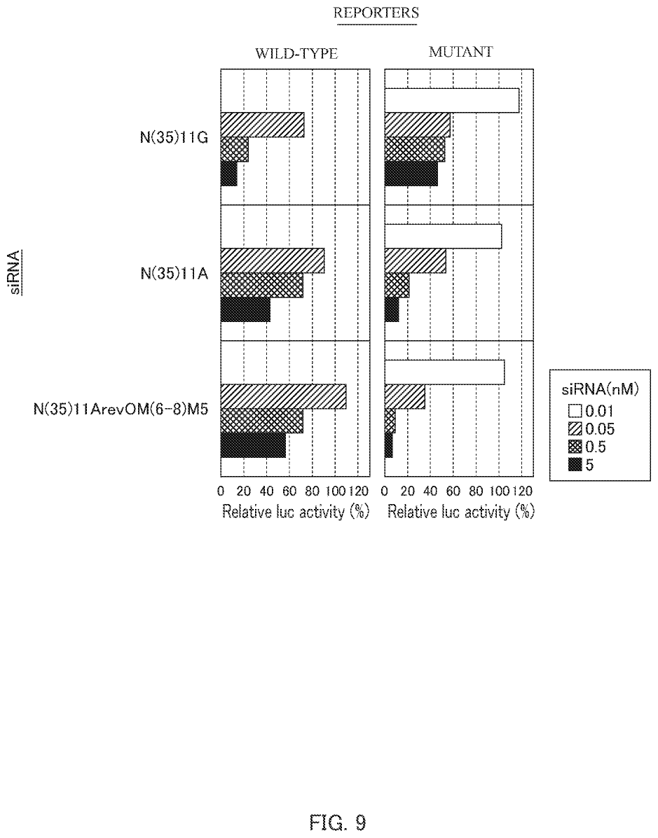

FIG. 9 shows gene silencing effects of the siRNAs.

N(35) 11G effectively suppressed the expression of the wild-type N allele more than that of the A-mutant N35 allele. In contrast, N(35)11A effectively suppressed the expression of the A-mutant N35 allele more than that of the wild-type N allele. N(35)11ArevOM(6-8)M5 had very weak silencing abilities to the wild-type allele and strong silencing abilities to the A-mutant N35 allele; as a result, specificity for the A-mutant N35 allele was increased.

### Example 2-2

This example, targeting the point mutation in nt 182 of N-ras cDNA shows that siRNAs suppress the expression of the G-mutant N182 allele more specifically than that of the wild-type allele of the N-ras (wt) gene (hereinafter, referred to as the “wild-type N allele”) by matching position 11 of each siRNA with the position of the point mutation in the G-mutant allele of the N-ras gene (c. 182A>G) (hereinafter, referred to as the “G-mutant N182 allele”), changing the base at the 5′-end of the guide strand of the siRNA from guanine to uracil, changing the base at the 5′-end of the passenger strand from adenine to guanine, replacing the group at the 2′-position of the pentose in each of ribonucleotides at positions 6-8 of the guide strand by OCH₃, and mismatching the base at position 5 of the guide strand with that of the G-mutant N182 allele.

First, as reporters for examining gene silencing effects, DNAs with the same nucleotide sequence as the G-mutant N182 allele were inserted into the 3′-UTR of the luciferase gene in an expression vector (psiCHECK) to construct a G-mutant N182 reporter. The sequences of the segments chemically synthesized and incorporated into the vector are indicated below.

| | |
|---|---|
| Wild-type N182 reporter: | |
| 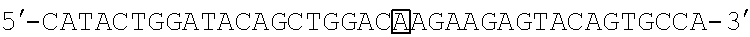 | (SEQ ID NO. 66) |
| 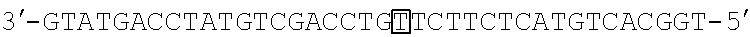 | (SEQ ID NO. 67) |
| G-mutant N182 reporter: | |
| 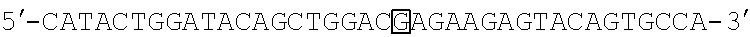 | (SEQ ID NO. 68) |
| 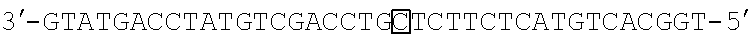 | (SEQ ID NO. 69) |

Double-stranded RNAs with the following sequences were chemically synthesized for siRNAs. N(182)11A has a sequence complementary to that of the wild-type N allele. N(182)11G is an siRNA in which position 11 corresponded to the position of the point mutation in the G-mutant N182 allele. N(182)11GrevOM(6-8)M5 is an siRNA in which position 11 corresponded to the position of the point mutation in the G-mutant N182 allele, the base at the 5′-end of the guide strand was changed from guanine to uracil, the base at the 5′-end of the passenger strand was changed from adenine to guanine, the group at the 2′-position of the pentose in each of ribonucleotides at positions 6-8 of the guide strand was replaced by OCH₃, and the base at position 5 of the siRNA's guide strand was mismatched with that of the G-mutant N182 allele. In the following sequences, the base pairs in the position corresponding to the position of the point mutation, the pairs of the modified bases at the 5′-ends of the guide and passenger strands, and the base pairs with the mismatched base are enclosed in rectangles. The nucleotides in which the group at the 2′-position of the pentose was replaced by OCH₃ are hatched.

| | |
|---|---|
| N(182)11A: | |
| 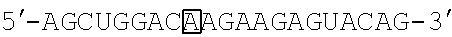 | (SEQ ID NO. 70) |
| 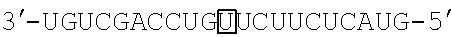 | (SEQ ID NO. 71) |
| N(182)11G: | |
| 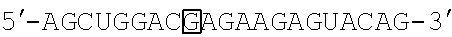 | (SEQ ID NO. 72) |
| 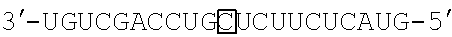 | (SEQ ID NO. 73) |
| N(182)11GrevOM(6-8)M5: | |
| 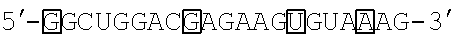 | (SEQ ID NO. 74) |
| 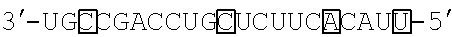 | (SEQ ID NO. 75) |

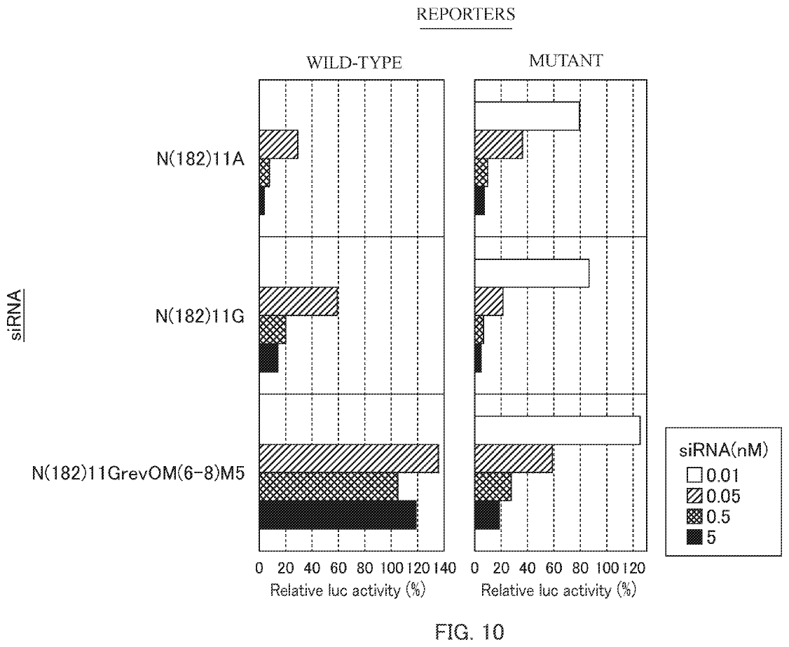

FIG. 10 shows gene silencing effects of the siRNAs.

N(182)11A effectively suppressed the expression of the wild-type N182 allele more than that of the G-mutant N182 allele. In contrast, N(182)11G effectively suppressed the expression of the A-mutant N182 allele more than that of the wild-type N182 allele. N(182) 11ArevOM(6-8)M5 lost silencing abilities to the wild-type allele and exhibited slightly reduced silencing abilities to the G-mutant N182 allele, resulting in a higher specificity for the G-mutant N182 allele.

### Example 3

In this example, various genes listed in Table 2 were used as the target genes to be silenced to show that siRNAs designed according to the method disclosed herein suppress the expression of the mutant alleles but do not suppress the expression of the wild-type alleles.

First, as reporters for examining gene silencing effects, DNAs with the same nucleotide sequences as wild-type (wt) and mutant alleles were chemically synthesized and inserted into the 3′-UTR of the luciferase gene in an expression vector (psiCHECK) to construct wild-type and mutant reporters, respectively. The sequences of the segments incorporated into the vectors are indicated in Table 2.

In the nucleotide sequences in the tables, lowercase letters denote sequences for ligating to a vector, and capital letters denote sequences derived from a gene. The parentheses in the K-ras gene indicate the type of mutation, the parentheses in the HTT gene indicate the position in the genome, and the parentheses in other genes indicate a place counted from the translation start site (i.e., A in the start codon ATG). “P” stands for a passenger strand, and “G” stands for a guide strand.

TABLE 2

| Gene Name | | | | | |
|---|---|---|---|---|---|
| (Position of | | | | | |
| mutation) | Function | Strand | Name | Sequence | Seq. ID No. |
| HTT | Wild-type | S | HTT_363125_WT_s | tcgagGTTAAGAGATCCCCACAGTACTTCAACGCTAGAAGAACACAg | 76 |
| (rs363125) | reporter | AS | HTT_363125_WT_as | aattcTGTGTTCTTCTAGCGTTGAAGTACTGTGGGGATCTCTTAACc | 77 |
| | Mutant | S | HTT_363125_SNP_s | tcgagGTTAAGAGATCCCCACAGTAATTCAACGCTAGAAGAACACAg | 78 |
| | reporter | AS | HTT_363125_SNP_as | aattcTGTGTTCTTCTAGCGTTGAATTACTGTGGGGATCTCTTAACc | 79 |
| HTT | Wild-type | S | HTT_362307_WT_s | tcgagCCGGAGCCTTTGGAAGTCTGCGCCCTTGTGCCCTGCCTCCAg | 80 |
| (rs362307) | reporter | AS | HTT_362307_WT_as | aattcTGGAGGCAGGGCACAAGGGCGCAGACTTCCAAAGGCTCCGGc | 81 |
| | Mutant | S | HTT_362307_SNP_s | tcgagCCGGAGCCTTTGGAAGTCTGTGCCCTTGTGCCCTGCCTCCAg | 82 |
| | reporter | AS | HTT_362307_SNP_as | aattcTGGAGGCAGGGCACAAGGGCACAGACTTCCAAAGGCTCCGGc | 83 |
| HTT | Wild-type | S | HTT_362331_WT_s | tcgagCCCACGCCTGCTCCCTCATCTACTGTGTGCACTTCATCCTGg | 84 |
| (rs362331) | reporter | AS | HTT_362331_WT_as | aattcCAGGATGAAGTGCACACAGTAGATGAGGGAGCAGGCGTGGGc | 85 |
| | Mutant | S | HTT_362331_SNP_s | tcgagCCCACGCCTGCTCCCTCATCCACTGTGTGCACTTCATCCTGg | 86 |
| | reporter | AS | HTT_362331_SNP_as | aattcCAGGATGAAGTGCACACAGTGGATGAGGGAGCAGGCGTGGGc | 87 |
| ATXN3 | Wild-type | S | ATXN3_WT_s | tcgagAGCAGCAGCAGCAGCAGCAGGGGGACCTATCAGGACAGAGTg | 88 |
| | reporter | AS | ATXN3_WT_as | aattcACTCTGTCCTGATAGGTCCCCCTGCTGCTGCTGCTGCTGCTc | 89 |
| | Mutant | S | ATXN3_SNP_s | tcgagAGCAGCAGCAGCAGCAGCAGCGGGACCTATCAGGACAGAGTg | 90 |
| | reporter | AS | ATXN3_SNP_as | aattcACTCTGTCCTGATAGGTCCCGCTGCTGCTGCTGCTGCTGCTc | 91 |
| ZMYM3 | Wild-type | S | ZMYM3_WT_Sense | tcgagTGCTGGAGACCATCCACTGGCGTGGGCAGATCCGTCATTTCg | 92 |
| | reporter | AS | ZMYM3_WT_Anti-sense | aattcGAAATGACGGATCTGCCCACGCCAGTGGATGGTCTCCAGCAc | 93 |
| | Mutant | S | ZMYM3_2206_Sense | tcgagTGCTGGAGACCATCCACTGGTGTGGGCAGATCCGTCATTTCg | 94 |
| | reporter | AS | ZMYM3_2206_Anti-sense | aattcGAAATGACGGATCTGCCCACAGCAGTGGATGGTCTCCAGCAc | 95 |
| CTNNB1 | Wild-type | S | CTNNB1_WT_Sense | tcgagGAATCCATTCTGGTGCCACTACCACAGCTCCTTCTCTGAGTg | 96 |
| | reporter | AS | CTNNB1_WT_Anti-sense | aattcACTCAGAGAAGGAGCTGTGGTAGTGGCACCAGAATGGATTCc | 97 |
| | Mutant | S | CTNNB1_121_Sense | tcgagGAATCCATTCTGGTGCCACTGCCACAGCTCCTTCTCTGAGTg | 98 |
| | reporter | AS | CTNNB1_121_Anti-sense | aattcACTCAGAGAAGGAGCTGTGGCAGTGGCACCAGAATGGATTCc | 99 |
| SMARCA4 | Wild-type | S | SMARCA4_WT_Sense | tcgagACCCCGCCGCCTGCTGCTGACGGGCACACCGCTGCAGAACAg | 100 |
| | reporter | AS | SMARCA4_WT_Anti-sense | aattcTGTTCTGCAGCGGTGTGCCCGTCAGCAGCAGGCGGCGGGGTc | 101 |
| | Mutant | S | SMARCA4_2729_Sense | tcgagACCCCGCCGCCTGCTGCTGATGGGCACACCGCTGCAGAACAg | 102 |
| | reporter | AS | SMARCA4_2729_Anti-sense | aattcTGTTCTGCAGCGGTGTGCCCATCAGCAGCAGGCGGCGGGGTc | 103 |
| SMO | Wild-type | S | SMO_WT_Sense | tcgagTGGCCCCAATCGGCCTGGTGCTCATCGTGGGAGGCTACTTCg | 104 |
| | reporter | AS | SMO_WT_Anti-sense | aattcGAAGTAGCCTCCCACGATGAGCACCAGGCCGATTGGGGCCAc | 105 |
| | Mutant | S | SMO_1234_Sense | tcgagTGGCCCCAATCGGCCTGGTGTTCATCGTGGGAGGCTACTTCg | 106 |
| | reporter | AS | SMO_1234_Anti-sense | aattcGAAGTAGCCTCCCACGATGAACACCAGGCCGATTGGGGCCAc | 107 |
| AR | Wild-type | S | AR_WT_Sense | tcgagCAAGGCCTTGCCTGGCTTCCGCAACTTACACGTGGACGACCg | 108 |
| | reporter | AS | AR_WT_Anti-sense | aattcGGTCGTCCACGTGTAAGTTGCGGAAGCCAGGCAAGGCCTTGc | 109 |
| | Mutant | S | AR_2180_Sense | tcgagCAAGGCCTTGCCTGGCTTCCTCAACTTACACGTGGACGACCg | 110 |
| | reporter | AS | AR_2180_Anti-sense | aattcGGTCGTCCACGTGTAAGTTGAGGAAGCCAGGCAAGGCCTTGc | 111 |
| DNM2 | Wild-type | S | DNM2_WT_Sense | tcgagGAATCAATCGCATCTTCCACGAGCGGTTCCCATTTGAGCTGg | 112 |
| | reporter | AS | DNM2_WT_Anti-sense | aattcCAGCTCAAATGGGAACCGCTCGTGGAAGATGCGATTGATTCc | 113 |
| | Mutant | S | DNM2_1102_Sense | tcgagGAATCAATCGCATCTTCCACAAGCGGTTCCCATTTGAGCTGg | 114 |
| | reporter | AS | DNM2_1102_Anti-sense | aattcCAGCTCAAATGGGAACCGCTTGTGGAAGATGCGATTGATTCc | 115 |
| KRT14 | Wild-type | S | KRT14_WT_Sense | tcgagTCAAGACCCTCAACAACAAGTTTGCCTCCTTCATCGACAAGg | 116 |
| | reporter | AS | KRT14_WT_Anti-sense | aattcCTTGTCGATGAAGGAGGCAAACTTGTTGTTGAGGGTCTTGAc | 117 |
| | Mutant | S | KRT14_520_Sense | tcgagTCAAGACCCTCAACAACAAGGTTGCCTCCTTCATCGACAAGg | 118 |
| | reporter | AS | KRT14_520_Anti-sense | aattcCTTGTCGATGAAGGAGGCAACCTTGTTGTTGAGGGTCTTGAc | 119 |
| IL4R | Wild-type | S | IL4R_WT_Sense | tcgagTCTCCGAAGCCCACACGTGTATCCCTGAGAACAACGGAGGCg | 120 |
| (223) | reporter | AS | IL4R_WT_Anti-sense | aattcGCCTCCGTTGTTCTCAGGGATACACGTGTGGGCTTCGGAGAc | 121 |
| | Mutant | S | IL4R_223_Sense | tcgagTCTCCGAAGCCCACACGTGTGTCCCTGAGAACAACGGAGGCg | 122 |
| | reporter | AS | IL4R_223_Anti-sense | aattcGCCTCCGTTGTTCTCAGGGACACACGTGTGGGCTTCGGAGAc | 123 |
| IL4R | Wild-type | S | IL4R_WT_Sense | tcgagCTTACCGCAGCTTCAGCAACTCCCTGAGCCAGTCACCGTGTg | 124 |
| (1507) | reporter | AS | IL4R_WT_Anti-sense | aattcACACGGTGACTGGCTCAGGGAGTTGCTGAAGCTGCGGTAAGc | 125 |
| | Mutant | S | IL4R_1507_Sense | tcgagCTTACCGCAGCTTCAGCAACCCCCTGAGCCAGTCACCGTGTg | 126 |
| | reporter | AS | IL4R_1507_Anti-sense | aattcACACGGTGACTGGCTCAGGGGGTTGCTGAAGCTGCGGTAAGc | 127 |
| MAPT | Wild-type | S | MAPT_WT_Sense | tcgagGGATAATATCAAACACGTCCCGGGAGGCGGCAGTGTGCAAAg | 128 |
| (1853) | reporter | AS | MAPT_WT_Anti-sense | aattcTTTGCACACTGCCGCCTCCCGGGACGTGTTTGATATTATCCc | 129 |
| | Mutant | S | MAPT_1853_Sense | tcgagGGATAATATCAAACACGTCCTGGGAGGCGGCAGTGTGCAAAg | 130 |
| | reporter | AS | MAPT_1853_Anti-sense | aattcTTTGCACACTGCCGCCTCCCAGGACGTGTTTGATATTATCCc | 131 |
| MS4A2 | Wild-type | S | MS4A2_WT_Sense | tcgagTGAGTTGGAAGACCCAGGGGAAATGTCTCCTCCCATTGATTg | 132 |
| | reporter | AS | MS4A2_WT_Anti-sense | aattcAATCAATGGGAGGAGACATTTCCCCTGGGTCTTCCAACTCAc | 133 |
| | Mutant | S | MS4A2_710_Sense | tcgagTGAGTTGGAAGACCCAGGGGGAATGTCTCCTCCCATTGATTg | 134 |
| | reporter | AS | MS4A2_710_Anti-sense | aattcAATCAATGGGAGGAGACATTCCCCCTGGGTCTTCCAACTCAc | 135 |
| PABPN1 | Wild-type | S | PABPN1_WT_Sense | tcgagGGCGGCGGCAGCAGCAGCGGGGGCTGCGGGCGGTCGGGGCTg | 136 |
| | reporter | AS | PABPN1_WT_Anti-sense | aattcAGCCCCGACCGCCCGCAGCCCCCGCTGCTGCTGCCGCCGCCc | 137 |
| | Mutant | S | PABPN1_35_Sense | tcgagGGCGGCGGCAGCAGCAGCGGCGGCTGCCGGGCGGTCGGGGTg | 138 |
| | reporter | AS | PABPN1_35_Anti-sense | aattcAGCCCCGACCGCCCGCAGCCGCCGCTGCTGCTGCCGCCGCCc | 139 |
| RHO | Wild-type | S | RHO_WT_Sense | tcgagGACGGGTGTGGTACGCAGCCCCTTCGAGTACCCACAGTACTg | 140 |
| (68) | reporter | AS | RHO_WT_Anti-sense | aattcAGTACTGTGGGTACTCGAAGGGGCTGCGTACCACACCCGTCc | 141 |
| | Mutant | S | RHO_68_Sense | tcgagGACGGGTGTGGTACGCAGCCACTTCGAGTACCCACAGTACTg | 142 |
| | reporter | AS | RHO_68_Anti-sense | aattcAGTACTGTGGGTACTCGAAGTGGCTGCGTACCACACCCGTCc | 140 |
| RHO | Wild-type | S | RHO_WT_Sense | tcgagCGGAGACGAGCCAGGTGGCCCCGGCCTAAGACCTGCCTAGGg | 141 |
| (1039) | reporter | AS | RHO_WT_Anti-sense | aattcCCTAGGCAGGTCTTAGGCCGGGGCCACCTGGCTCGTCTCCGc | 143 |
| | Mutant | S | RHO_1039_Sense | tcgagCGGAGACGAGCCAGGTGGCCTCGGCCTAAGACCTGCCTAGGg | 144 |
| | reporter | AS | RHO_1039_Anti-sense | aattcCCTAGGCAGGTCTTAGGCCGAGGCCACCTGGCTCGTCTCCGc | 145 |
| SCNIA | Wild-type | S | SCN1A_WT_Sense | tcgagTGAGAACATTCAGAGTTCTCCGAGCATTGAAGACGATTTCAg | 146 |
| | reporter | AS | SCN1A_WT_Anti-sense | aattcTGAAATCGTCTTCAATGCTCGGAGAACTCTGAATGTTCTCAc | 147 |
| | Mutant | S | SCN1A_664_Sense | tcgagTGAGAACATTCAGAGTTCTCTGAGCATTGAAGACGATTTCAg | 148 |
| | reporter | AS | SCN1A_664_Anti-sense | aattcTGAAATCGTCTTCAATGCTCAGAGAACTCTGAATGTTCTCAc | 149 |
| APOB | Wild-type | S | APOB_WT_Sense | tcgagCTTGAATTCCAAGAGCACACGGTCTTCAGTGAAGCTGCAGGg | 150 |
| | reporter | AS | APOB_WT_Anti-sense | aattcCCTGCAGCTTCACTGAAGACCGTGTGCTCTTGGAATTCAAGc | 151 |
| | Mutant | S | APOB_10580_Sense | tcgagCTTGAATTCCAAGAGCACACAGTCTTCAGTGAAGCTGCAGGg | 152 |
| | reporter | AS | APOB_10580_Anti-sense | aattcCCTGCAGCTTCACTGAAGACTGTGTGCTCTTGGAATTCAAGc | 153 |
| F12 (CG) | Wild-type | S | F12_WT_Sense | tcgagACCGCCGAAGCCTCAGCCCACGACCCGGACCCCGCCTCAGTg | 154 |
| | reporter | AS | F12_WT_Anti-sense | aattcACTGAGGCGGGGTCCGGGTCGTGGGCTGAGGCTTCGGCGGTc | 155 |
| | Mutant | S | F12_983_CG_Sense | tcgagACCGCCGAAGCCTCAGCCCAGGACCCGGACCCCGCCTCAGTg | 156 |
| | reporter | AS | F12_983_CG_Anti-sense | aattcACTGAGGCGGGGTCCGGTCCTGGGCTGAGGCTTCGGCGGTCc | 157 |
| CLCN7 | Wild-type | S | CLCN7_WT_Sense | tcgagTGTTCCGGGCCCTGGGCCTGCGGCACCTGGTGGTGGTGGACg | 158 |
| | reporter | AS | CLCN7_WT_Anti-sense | aattcGTCCACCACCACCAGGTGCCGCAGGCCCAGGGCCCGGAACAc | 159 |
| | Mutant | S | CLCN7_2299_Sense | tcgagTGTTCCGGGCCCTGGGCCTGTGGCACCTGGTGGTGGTGGACg | 160 |
| | reporter | AS | CLCN7_2299_Anti-sense | aattcGTCCACCACCACCAGGTGCCACAGGCCCAGGGCCCGGAACAc | 161 |
| SCN8A | Wild-type | S | SCN8A_WT_Sense | tcgagTCCCCTCCATCATGAATGTGCTGCTGGTGTGTCTCATCTTCg | 162 |
| | reporter | AS | SCN8A_WT_Anti-sense | aattcGAAGATGAGACACACCAGCAGCACATTCATGATGGAGGGGAc | 163 |
| | Mutant | S | SCN8A_3991_Sense | tcgagTCCCCTCCATCATGAATGTGGTGCTGGTGTGTCTCATCTTCg | 164 |
| | reporter | AS | SCN8A_3991_Anti-sense | aattcGAAGATGAGACACACCAGCACCACATTCATGATGGAGGGGAc | 165 |
| PCSK9 | Wild-type | S | PCSK9_WT_Sense | tcgagACATCATTGGTGCCTCCAGCGACTGCAGCACCTGCTTTGTGg | 166 |
| | reporter | AS | PCSK9_WT_Anti-sense | aattcCACAAAGCAGGTGCTGCAGTCGCTGGAGGCACCAATGATGTc | 167 |
| | Mutant | S | PCSK9_1120_Sense | tcgagACATCATTGGTGCCTCCAGCTACTGCAGCACCTGCTTTGTGg | 168 |
| | reporter | AS | PCSK9_1120_Anti-sense | aattcCACAAAGCAGGTGCTGCAGTAGCTGGAGGCACCAATGATGTc | 169 |
| KRT6A | Wild-type | S | KRT6A_WT_Sense | tcgagTCAAGACCCTCAACAACAAGTTTGCCTCCTTCATCGACAAGg | 170 |
| | reporter | AS | KRT6A_WT_Anti-sense | aattcCTTGTCGATGAAGGAGGCAAACTTGTTGTTGAGGGTCTTGAc | 171 |
| | Mutant | S | KRT6A_520_Sense | tcgagTCAAGACCCTCAACAACAAGGTTGCCTCCTTCATCGACAAGg | 172 |
| | reporter | AS | KRT6A_520_Anti-sense | aattcCTTGTCGATGAAGGAGGCAACCTTGTTGTTGAGGGTCTTGAc | 173 |

For siRNAs, the base at position 11 of each gene corresponded to the position of the point mutation in the corresponding mutant allele, the group at the 2′-position of the pentose in each of the ribonucleotides at positions 6-8 of the siRNA's guide strand was replaced by OCH₃, and the base at position 6 of the guide strand was mismatched with that of the corresponding mutant allele. The 5′-ends of the guide and passenger strands of each siRNA were replaced as indicated in Table 3.

TABLE 3

| Gene Name | | |
|---|---|---|
| (Position of | Base change | Base change |
| mutation) | at 5′-end of G strand | at 5′-end of P strand |
| HTT (rs363125) | — | C→G |
| HTT (rs362307) | G→U | — |
| HTT (rs362331) | — | C→G |
| ATXN3 | G→U | A→G |
| ZMYM3 | A→U | U→G |
| CTNNB1 | G→U | — |
| SMARCA4 | C→U | — |
| SMO | C→G | — |
| AR | G→U | U→G |
| DNM2 | G→U | U→G |
| KRT14 | G→U | — |
| IL4R (223) | — | A→G |
| IL4R (1507) | — | U→G |
| MAPT | G→U | U→G |
| MS4A2 | A→U | C→G |
| PABPN1 | G→U | A→G |
| RHO(68) | G→U | A→G |
| RHO (1039) | — | A→G |
| SCNIA | — | — |
| APOB | C→U | — |
| F12 | G→U | U→G |
| CLCN7 | A→U | U→G |
| SCN8A | C→U | U→G |
| PCSK9 | G→U | C→G |
| KRT6A | A→U | A→G |

The reporters indicated in Table 2 were used for the corresponding genes. and reporter assays were performed using siRNAs indicated in Tables 4 and 5 using the aforementioned method.

TABLE 4

| Gene Name | | | | |
|---|---|---|---|---|
| (Position of | | | | Seq. |
| mutation) | Name | Strand | Sequence | ID No. |
| HTT | SiHTT_363125-WT | P | CCACAGUACUUCAACGCUAGA | 174 |
| (rs363125) | | G | UAGCGUUGAAGUACUGUGGGG | 175 |
| HTT | SiHTT_362307-WT | P | GAAGUCUGCGCCCUUGUGCCC | 176 |
| (rs362307) | | G | GCACAAGGGCGCAGACUUCCA | 177 |
| HTT | SiHTT_362331-WT | P | CCCUCAUCUACUGUGUGCACU | 178 |
| (rs362331) | | G | UGCACACAGUAGAUGAGGGAG | 179 |
| ATXN3 | SiATXN3-WT | P | AGCAGCAGGGGGACCUAUCAG | 180 |
| | | G | GAUAGGUCCCCCUGCUGCUGC | 181 |
| ZMYM3 | SiZMYM3_WT | P | UCCACUGGCGUGGGCAGAUCC | 182 |
| | | G | AUCUGCCCACGCCAGUGGAUG | 183 |
| CTNNB1 | siCTNNB1_WT | P | GUGCCACUACCACAGCUCCUU | 184 |
| | | G | GGAGCUGUGGUAGUGGCACCA | 185 |
| SMARCA4 | siSMARCA4_WT | P | GCUGCUGACGGGCACACCGCU | 186 |
| | | G | CGGUGUGCCCGUCAGCAGCAG | 187 |
| SMO | siSMO_WT | P | GCCUGGUGCUCAUCGUGGGAG | 188 |
| | | G | CCCACGAUGAGCACCAGGCCG | 189 |

TABLE 5

| Gene Name | | | | |
|---|---|---|---|---|
| (Position of | | | | Seq. |
| mutation) | Name | Strand | Sequence | ID No. |
| HTT | SiHTT_363125-SNP | P | GCACAGUAAUUCAUCGCUAGA | 190 |
| (rs363125) | | G | UAGCGAUGAAUUACUGUGCGG | 191 |
| HTT | SiHTT_362307-SNP | P | GAAGUCUGUGCCCAUGUGACC | 192 |
| (rs362307) | | G | UCACAUGGGCACAGACUUCCA | 193 |
| HTT | SiHTT_362331-SNP | P | GCCUCAUCCACUGAGUGCACU | 194 |
| (rs362331) | | G | UGCACUCAGUGGAUGAGGCAG | 195 |
| ATXN3 | SiATXN3-SNP | P | GGCAGCAGCGGGAGCUAUAAG | 196 |
| | | G | UAUAGCUCCCGCUGCUGCCGC | 197 |
| ZMYM3 | SiZMYM3_MUT_2206 | P | GCCACUGGUGUGGCCAGAACC | 198 |
| | | G | UUCUGGCCACACCAGUGGCUG | 199 |
| CTNNB1 | SiCTNNB1_MUT_121 | P | GUGCCACUGCCACUGCUCAUU | 200 |
| | | G | UGAGCAGUGGCAGUGGCACCA | 201 |
| SMARCA4 | SiSMARCA4_MUT_2729 | P | GCUGCUGAUGGGCUCACCACU | 202 |
| | | G | UGGUGAGCCCAUCAGCAGCAG | 203 |
| SMO | siSMO_MUT_1234 | P | GCCUGGUGUUCAUGGUGGAAG | 204 |
| | | G | GCCACCAUGAACACCAGGCCG | 205 |
| AR | SiAR_MUT_2180 | P | GGGCUUCCUCAACAUACAAGU | 206 |
| | | G | UUGUAUGUUGAGGAAGCCCGG | 207 |
| DNM2 | siDNM2_MUT_1102 | P | GCUUCCACAAGCGCUUCCAAU | 208 |
| | | G | UGGAAGCGCUUGUGGAAGCUG | 209 |
| KRT14 | SiKRT14_MUT_1151 | P | GGAGCAGCCGGCCGAGCUACG | 210 |
| | | G | UAGCUCGGCCGGCUGCUCCUC | 211 |
| IL4R | SiILR4_MUT_223 | P | GCACGUGUGUCCCAGAGAACA | 212 |
| (223) | | G | UUCUCUGGGACACACGUGCGG | 213 |
| IL4R | SiILR4_MUT_1507 | P | GCAGCAACCCCCUCAGCCAGU | 214 |
| (1507) | | G | UGGCUGAGGGGGUUGCUGCAG | 215 |
| MAPT | siMAPT_MUT_1853 | P | GCACGUCCUGGGACGCGGAAG | 216 |
| (1853) | | G | UCCGCGUCCCAGGACGUGCUU | 217 |
| MS4A2 | siMS4A2_MUT_710 | P | GCCAGGGGGAAUGACUCCACC | 218 |
| | | G | UGGAGUCAUUCCCCCUGGCUC | 219 |
| PABPN1 | siPABPN1_MUT_35 | P | GGCAGCGGCGGCUCCGGGAGG | 220 |
| | | G | UCCCGGAGCCGCCGCUGCCGC | 221 |
| RHO | siRHO_MUT_68 | P | GCGCAGCCACUUCCAGUAACC | 222 |
| (68) | | G | UUACUGGAAGUGGCUGCGCAC | 223 |
| RHO | SiRHO_MUT_1039 | P | GGGUGGCCUCGGCGUAAGACC | 224 |
| (1039) | | G | UCUUACGCCGAGGCCACCCGG | 225 |
| SCNIA | siSCN1A_MUT_664 | P | GAGUUCUCUGAGCUUUGAAGA | 226 |
| | | G | UUCAAAGCUCAGAGAACUCUG | 227 |
| APOB | siAPOB_MUT_10580 | P | GAGCACACAGUCUACAGUAAA | 228 |
| | | G | UACUGUAGACUGUGUGCUCUU | 229 |
| F12 | siF12_MUT_983 CG | P | GCAGCCCAGGACCGGGACACC | 230 |
| (983 CG) | | G | UGUCCCGGUCCUGGGCUGCGG | 231 |
| CLCN7 | siCLCN7_MUT_2299 | P | GGACCCGGACGCCGUGGACCA | 323 |
| | | G | UCCAGCUGCCACAGGCCCCGG | 233 |
| SCN8A | siSCN8A_MUT_3991 | P | GGAAUGUGGUGCUCGUGUAUC | 234 |
| | | G | UACACGAGCACCACAUUCCUG | 235 |
| PCSK9 | siPCSK9_MUT_1120 | P | GCUCCAGCUACUGGAGCAACU | 236 |
| | | G | UUGCUCCAGUAGCUGGAGCCA | 237 |
| KRT6A | SiKRT6A_MUT_520 | P | GCAACAAGGUUGCGUCCUACA | 238 |
| | | G | UAGGACGCAACCUUGUUGCUG | 239 |

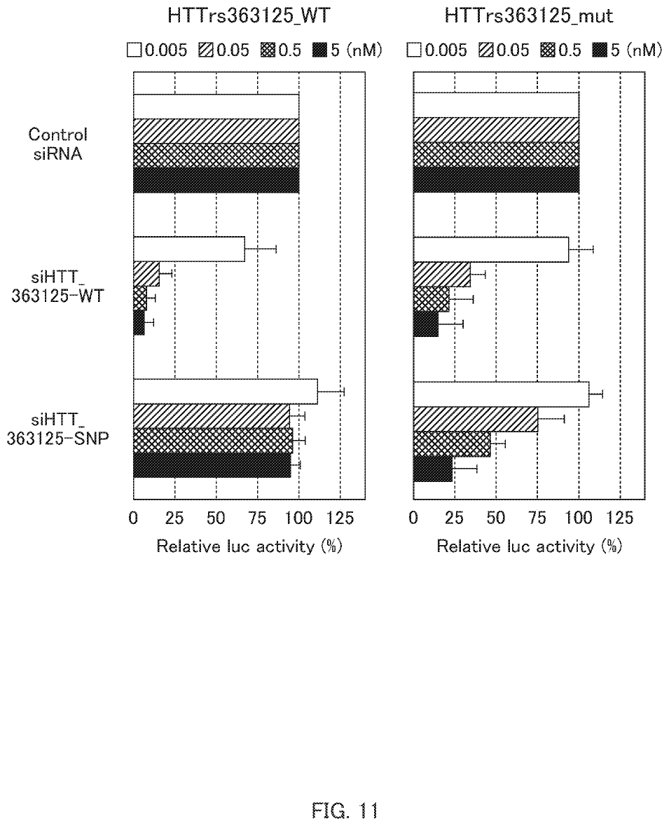

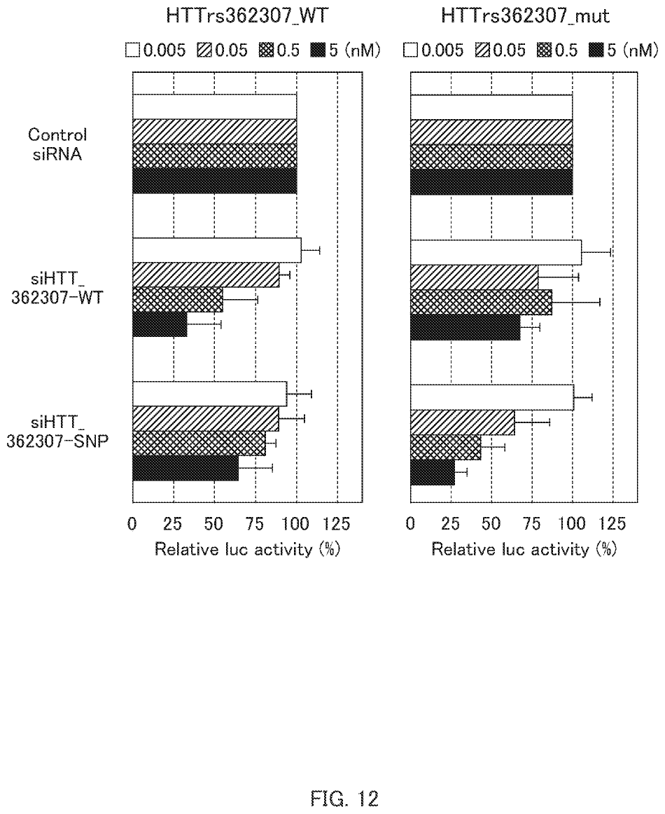

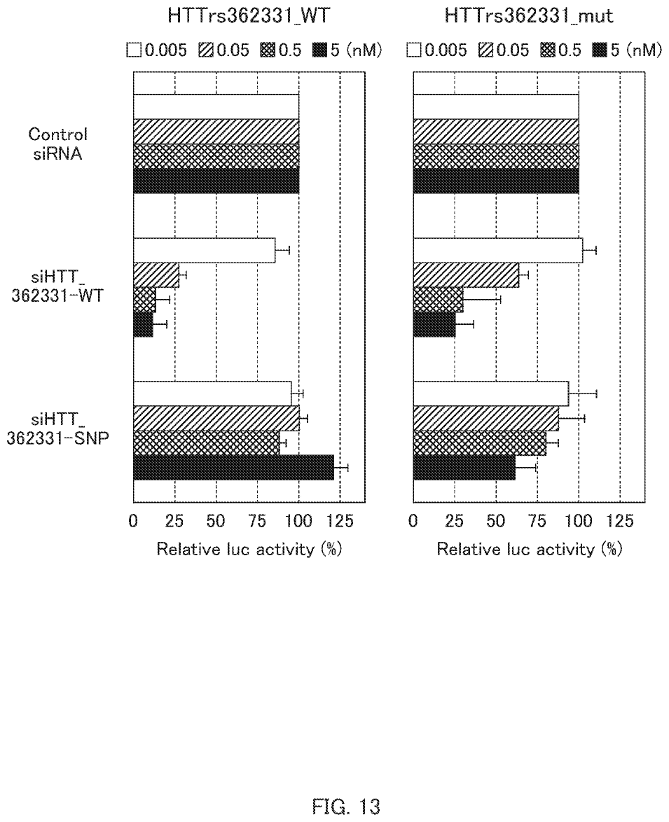

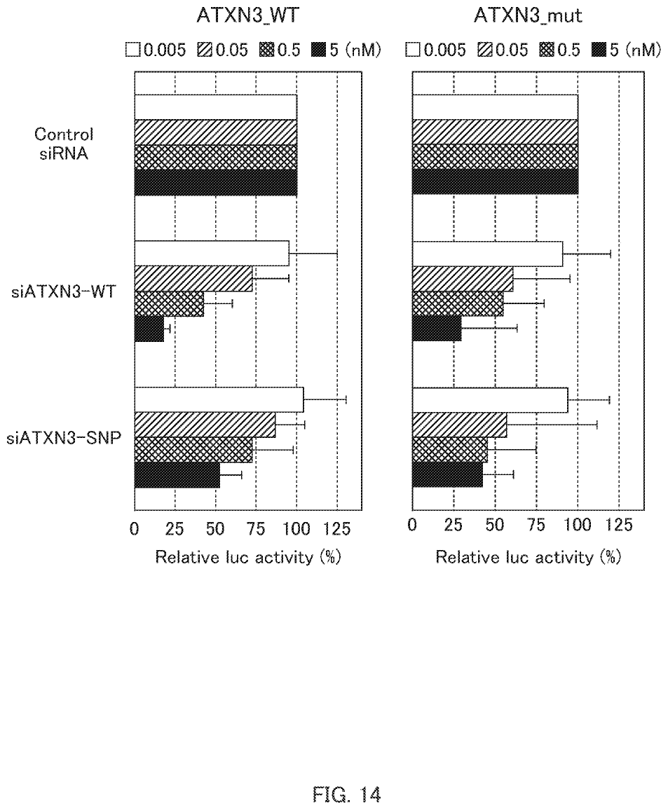

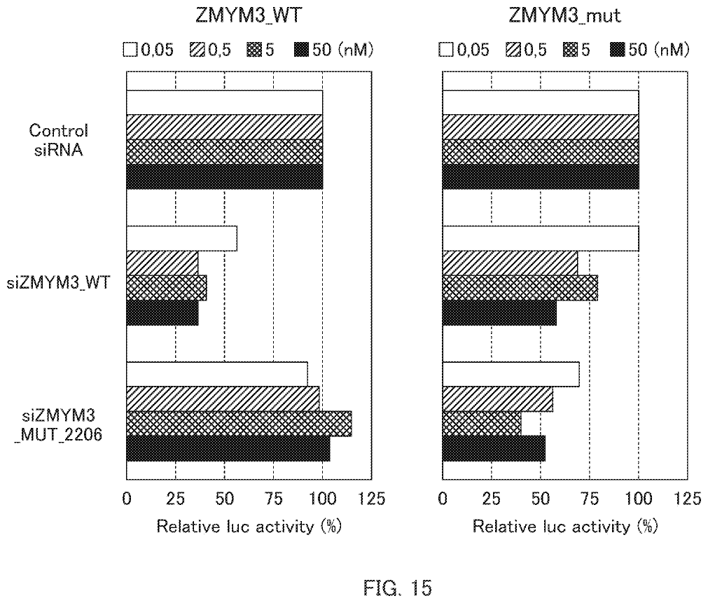

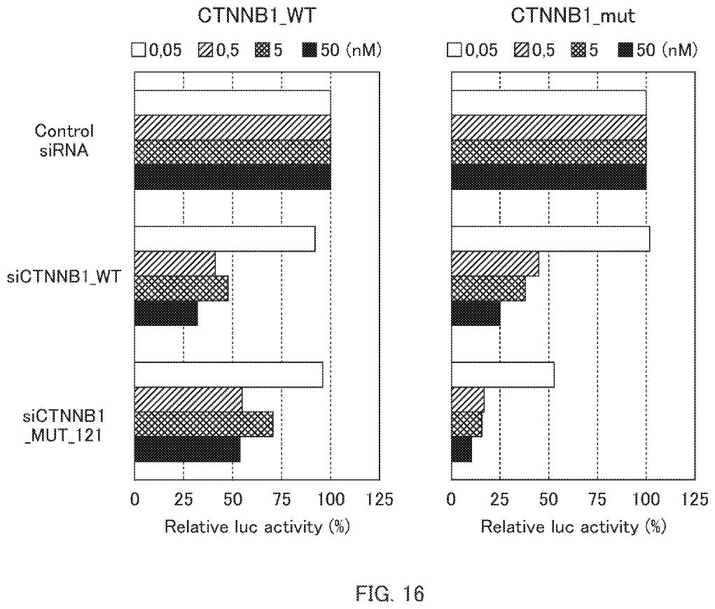

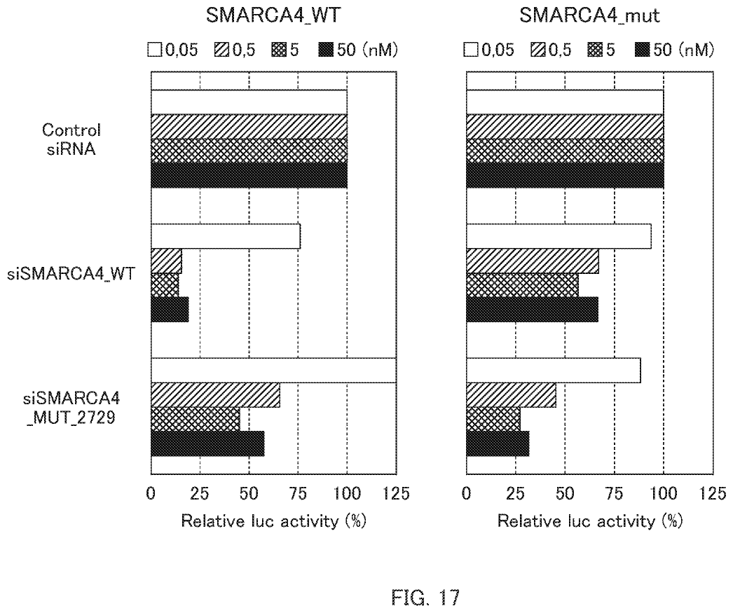

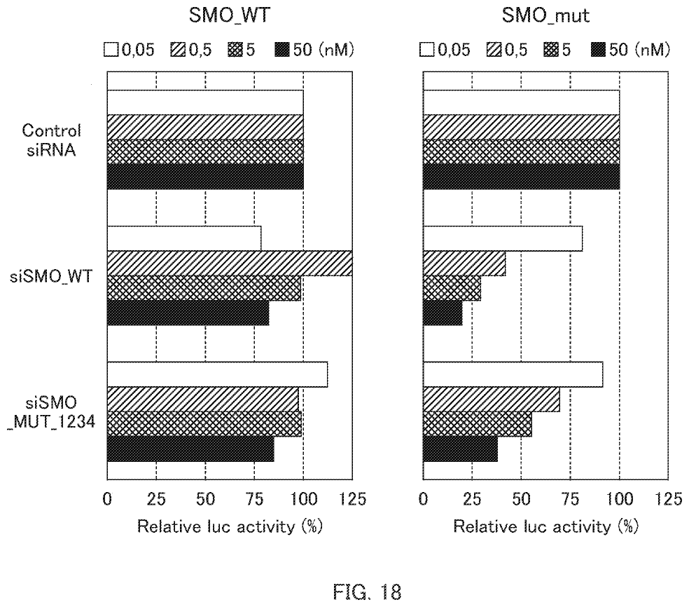

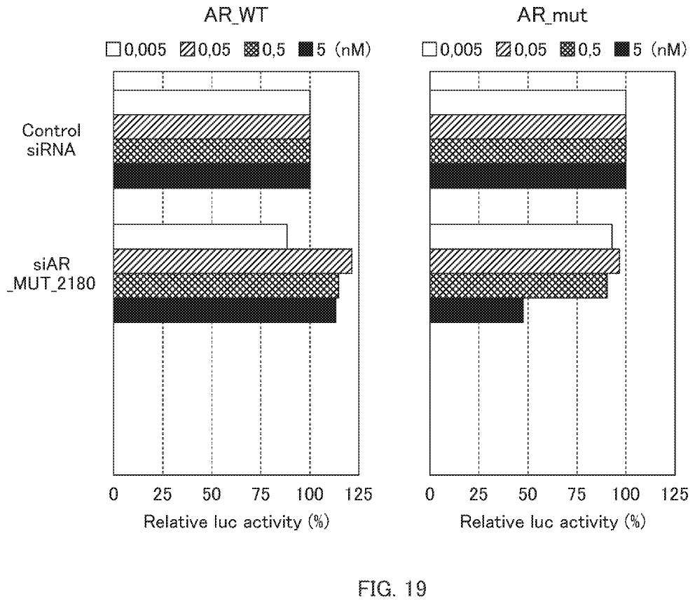

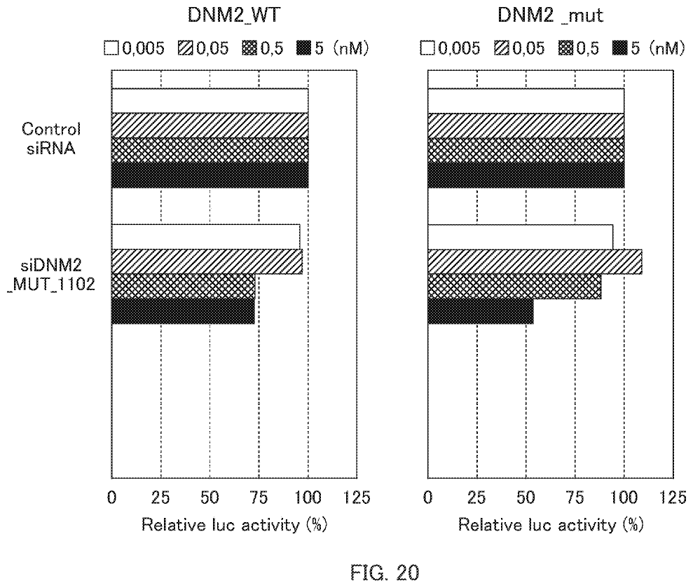

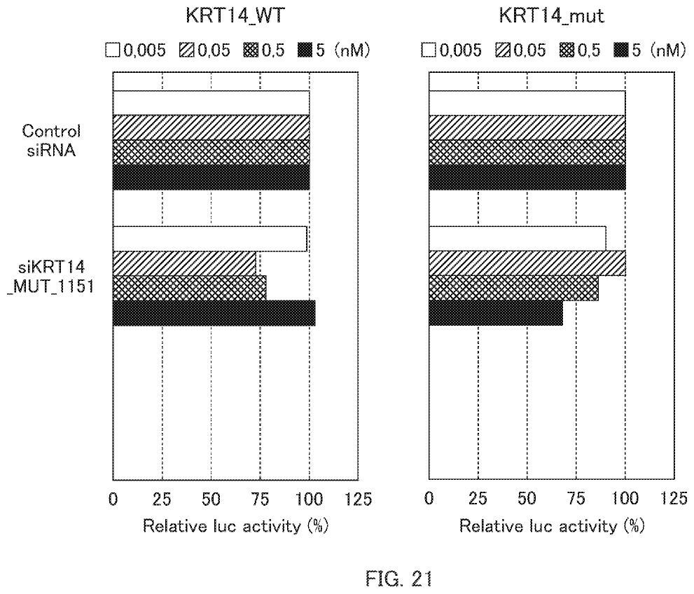

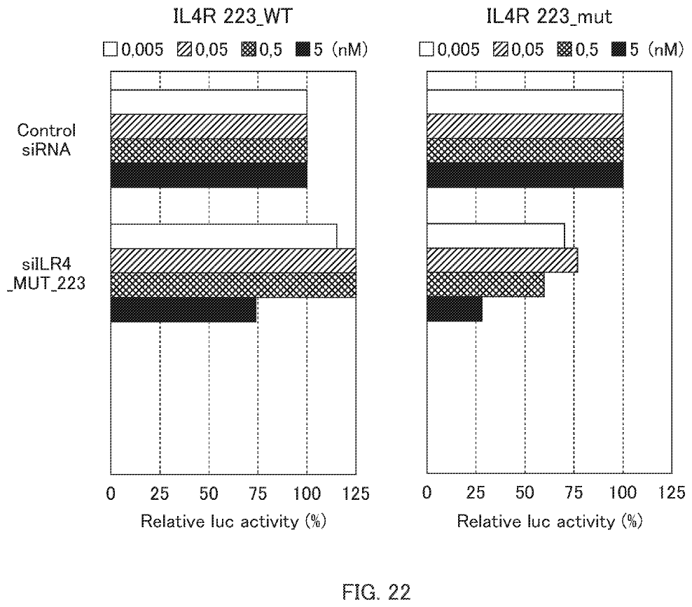

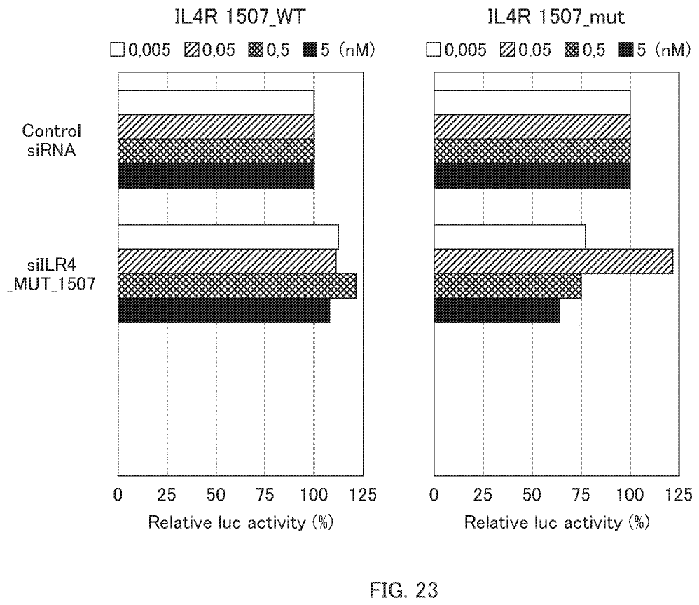

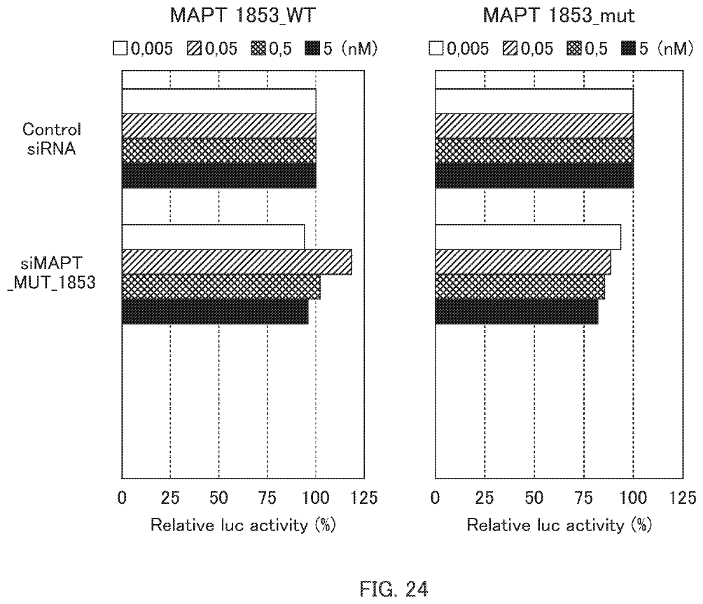

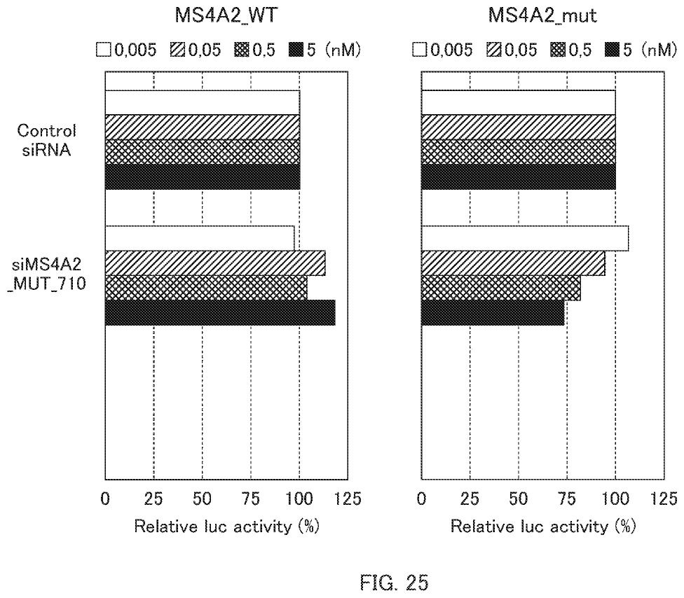

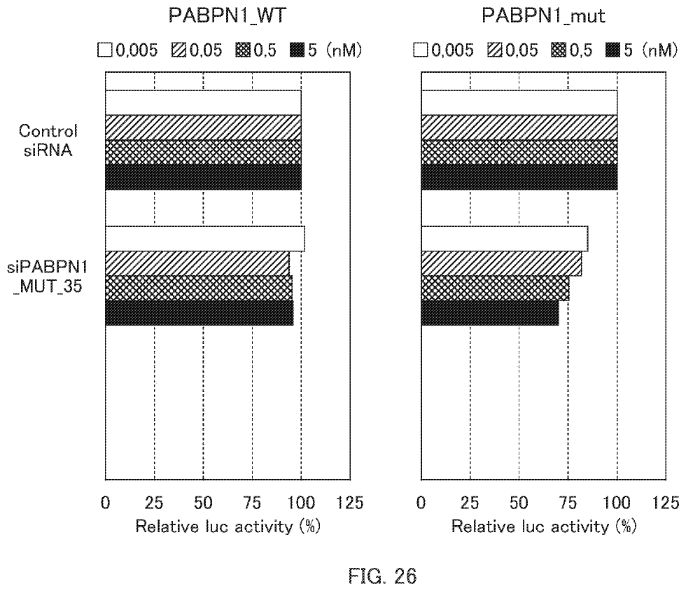

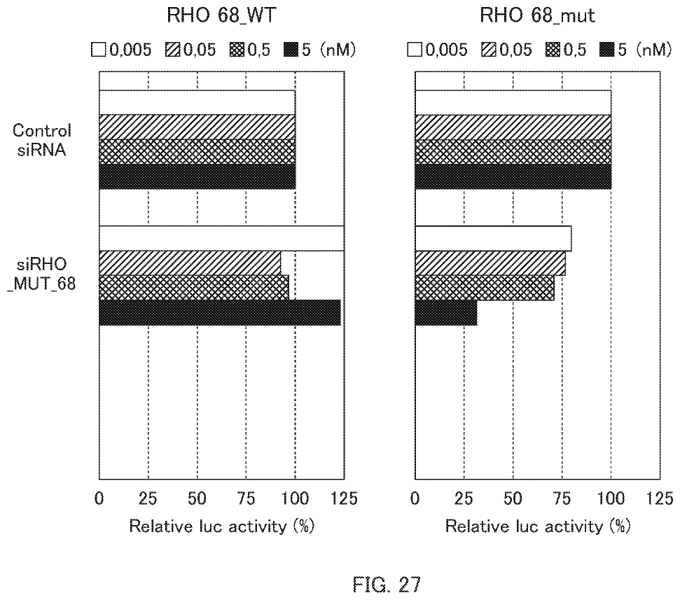

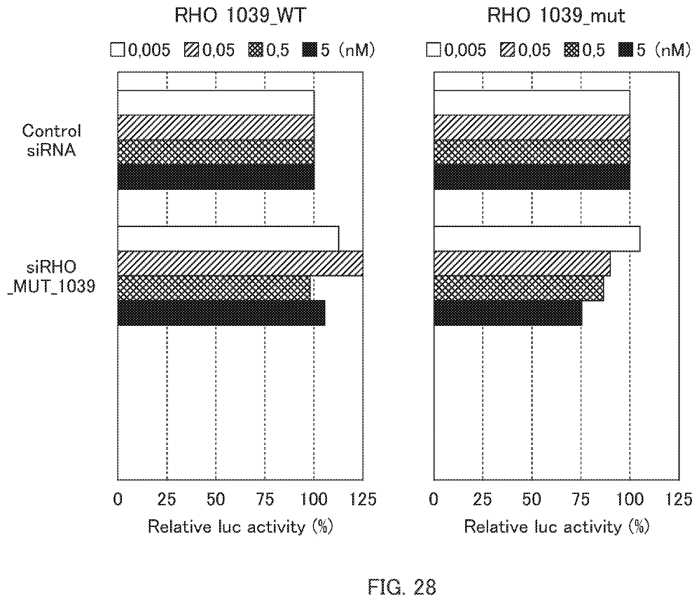

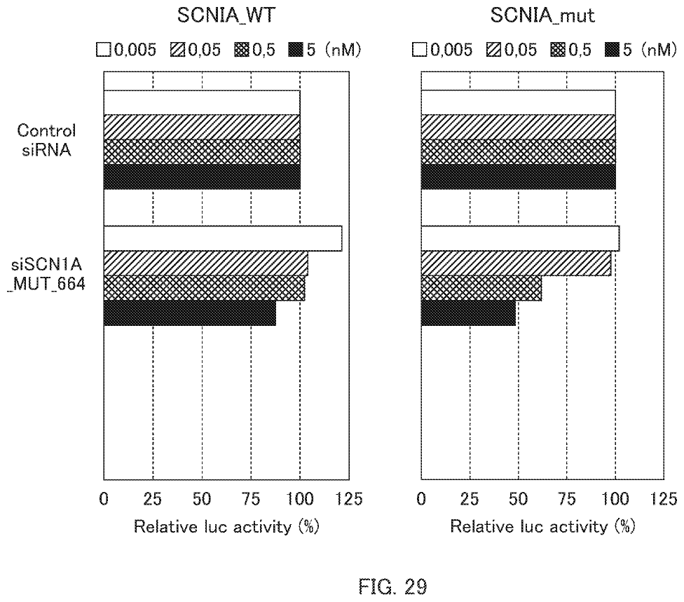

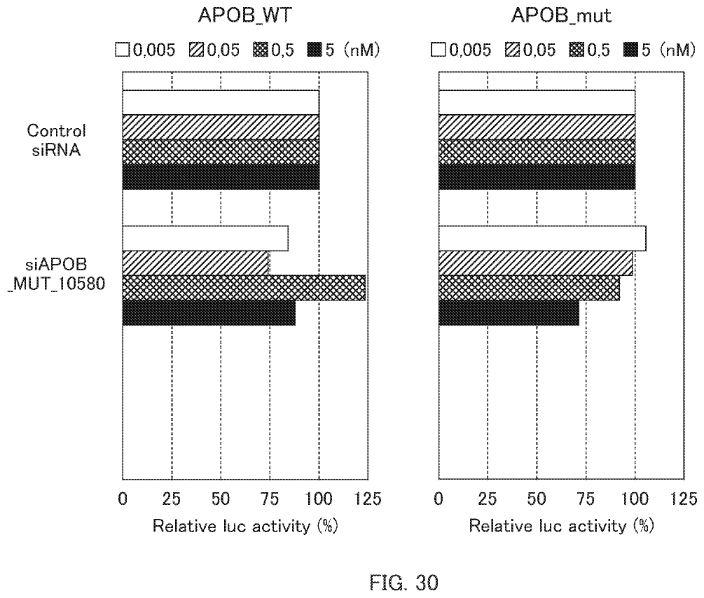

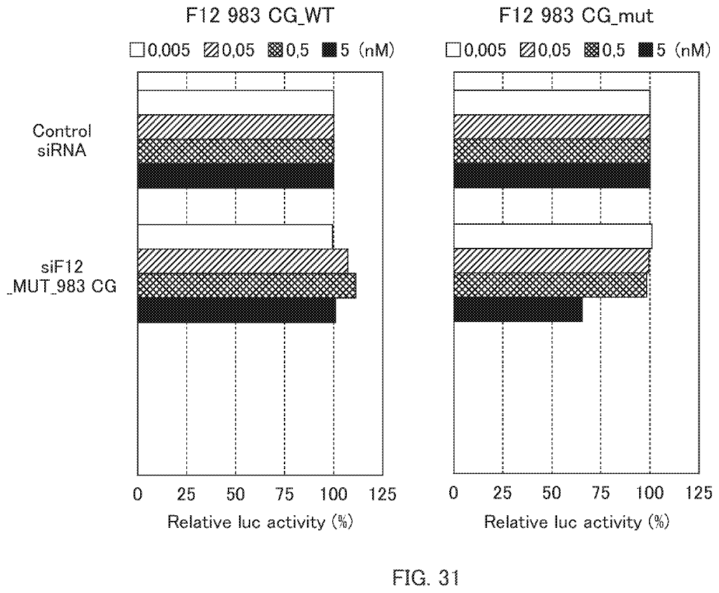

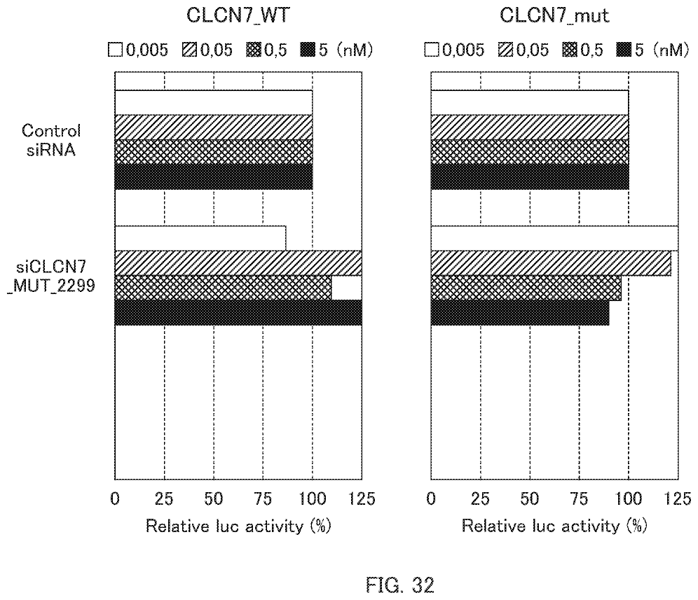

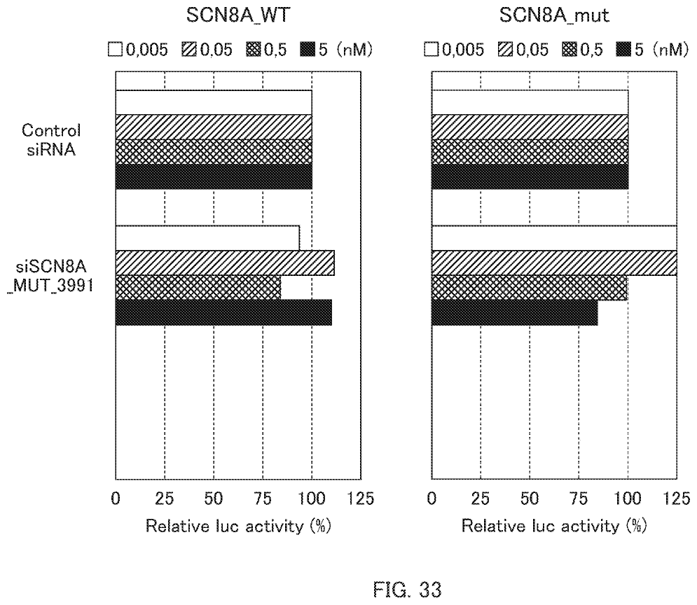

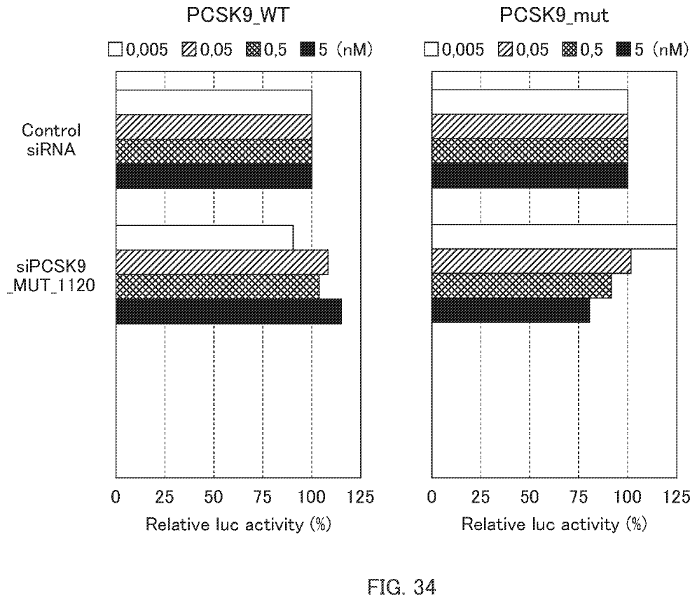

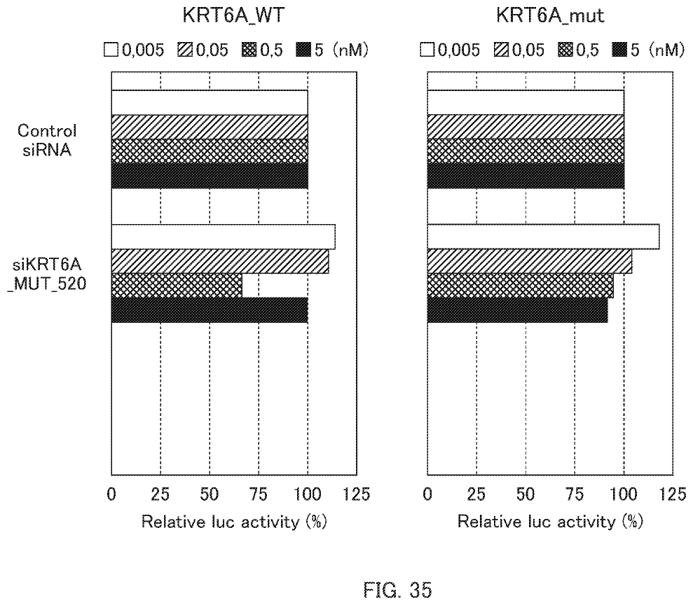

FIGS. 11 to 35 show the results of the assays. For all genes, the siRNAs suppressed the expressions of the mutant alleles in a concentration-dependent manner, though the expressions of the wild-type alleles were hardly suppressed.

### Industrial Applicability

The present invention allowed to provide novel RNA molecules, novel chimeric NA molecules, novel double-stranded RNA molecules, and novel double-stranded chimeric NA molecules.

1. An RNA molecule that targets a mutant allele of a gene, the mutant allele having a point mutation relative to a wild-type allele of the gene, wherein the RNA molecule satisfies the following:
(1) the molecule has a nucleotide sequence complementary to a nucleotide sequence of a coding region of the mutant allele except for a base specified in (2-1) below; and
(2) when counted from the base at the 5′-end of a nucleotide sequence complementary to the nucleotide sequence of the mutant allele,
(2-1) a base at position 5 or 6 is mismatched with a base in the mutant allele;
(2-2) a base at position 10 or 11 is at the position of the point mutation and is identical to the base at the position of the point mutation in the mutant allele;
(2-3) the group at the 2′-position of the pentose in the ribonucleotide at position 8 is modified with OCH₃, halogen, or LNA; and
(2-4) the group at the 2′-position of the pentose in the ribonucleotide at position 7 is not modified with any of OCH₃, halogen, and LNA.

2. The RNA molecule according to claim 1, wherein the group at the 2′-position of the pentose in the ribonucleotide at position 6, counted from the base at the 5′-end of the nucleotide sequence complementary to the nucleotide sequence of the mutant allele, is modified with OCH₃, halogen, or LNA.

3. The RNA molecule according to claim 1, wherein the ribonucleotide at position 7 is free from modification.

4. The RNA molecule according to claim 1, wherein the halogen is fluorine.

5. The RNA molecule according to claim 1, wherein, when the base at the 5′-end of the nucleotide sequence specified in (1) is cytosine or guanine, the base is replaced by adenine or uracil.

6. The RNA molecule according to claim 1, wherein, when the base at the 3′-end of the nucleotide sequence specified in (1) is adenine or uracil, it is replaced by cytosine or guanine.

7. The RNA molecule according to claim 1, wherein the RNA molecule comprises 13-28 nucleotides.

8. The RNA molecule according to claim 1, further comprising 1-3 nucleotide(s) at the 3′-end of the nucleotide sequence specified in (1).

9. A chimeric NA molecule that targets a mutant allele of a gene, the mutant allele having a point mutation relative to a wild-type allele of the gene, wherein the chimeric NA molecule satisfies the following:
(1) the molecule has a nucleotide sequence complementary to a nucleotide sequence of a coding region of the mutant allele except for a base specified in (2-1) below; and
(2) when counted from the base at the 5′-end of a nucleotide sequence complementary to the nucleotide sequence of the mutant allele,
(2-1) a base at position 5 or 6 is mismatched with a base in the mutant allele;
(2-2) a base at position 10 or 11 is at the position of the point mutation and is identical to the base at the position of the point mutation in the mutant allele;
(2-3) the group at the 2′-position of the pentose in the ribonucleotide at position 8 is modified with OCH₃, halogen, or LNA; and
(2-4) the group at the 2′-position of the pentose in the ribonucleotide at position 7 is not modified with any of OCH₃, halogen, and LNA, wherein the chimeric NA molecule comprises a ribonucleotide as well as a deoxyribonucleotide, an artificial nucleic acid, or a nucleic acid analog.

10. A double-stranded RNA molecule that targets a mutant allele of a gene, wherein the mutant allele has a point mutation relative to a wild-type allele of the gene, the double-stranded RNA molecule comprising a guide strand and a passenger strand, wherein the guide strand satisfies the following:
(1) the guide strand has a nucleotide sequence complementary to a nucleotide sequence of a coding region of the mutant allele except for a base specified in (2-1) below; and
(2) when counted from the base at the 5′-end of a nucleotide sequence complementary to the nucleotide sequence of the mutant allele,
(2-1) a base at position 5 or 6 is mismatched with a base in the mutant allele;
(2-2) a base at position 10 or 11 is at the position of the point mutation and is identical to the base at the position of the point mutation in the mutant allele;
(2-3) the group at the 2′-position of the pentose in the ribonucleotide at position 8 is modified with OCH₃, halogen, or LNA; and
(2-4) the group at the 2′-position of the pentose in the ribonucleotide at position 7 is not modified with any of OCH₃, halogen, and LNA.

11. The double-stranded RNA molecule according to claim 10, wherein the RNA molecule comprises an overhang at the 3′-end of the guide strand and/or an overhang at the 3′-end of the passenger strand.

12. The double-stranded RNA molecule according to claim 11, wherein either or both of the overhangs comprise 1-3 nucleotides.

13. A double-stranded chimeric NA molecule that targets a mutant allele of a gene, the mutant allele having a point mutation relative to a wild-type allele of the gene, the double-stranded chimeric RNA molecule comprising a guide strand and a passenger strand, the double-stranded chimeric RNA molecule comprising a ribonucleotide as well as a deoxyribonucleotide, an artificial nucleic acid, or a nucleic acid analog, wherein the guide strand satisfies the following:
(1) the guide strand has a nucleotide sequence complementary to a nucleotide sequence of a coding region of the mutant allele except for a base specified in (2-1) below; and
(2) when counted from the base at the 5′-end of a nucleotide sequence complementary to the nucleotide sequence of the mutant allele,
(2-1) a base at position 5 or 6 is mismatched with a base in the mutant allele:
(2-2) a base at position 10 or 11 is at the position of the point mutation and is identical to the base at the position of the point mutation in the mutant allele;
(2-3) the group at the 2′-position of the pentose in the ribonucleotide at position 8 is modified with OCH₃, halogen, or LNA; and
(2-4) the group at the 2′-position of the pentose in the ribonucleotide at position 7 is not modified with any of OCH₃, halogen, and LNA.

14. (canceled)

15. (canceled)

16. A method for performing RNA interference to target a mutant allele of a gene in a cell containing a wild-type allele of the gene and the mutant allele, wherein the mutant allele has a point mutation, the method comprising the step of:
introducing the RNA molecule of claim 1 into the cell.

17-18. (canceled)

19. A method for performing RNA interference to target a mutant allele of a gene in a cell containing a wild-type allele of the gene and the mutant allele, wherein the mutant allele has a point mutation relative to the wild-type allele, the method comprising the step of:
introducing the chimeric NA molecule of claim 9 into the cell.

20. A method for performing RNA interference to target a mutant allele of a gene in a cell containing a wild-type allele of the gene and the mutant allele, wherein the mutant allele has a point mutation relative to the wild-type allele, the method comprising the step of:
introducing the double-stranded RNA molecule of claim 10 into the cell.

21. A method for performing RNA interference to target a mutant allele of a gene in a cell containing a wild-type allele of the gene and the mutant allele, wherein the mutant allele has a point mutation relative to the wild-type allele, the method comprising the step of:
introducing the double-stranded chimeric NA molecule of claim 13 into the cell.
